(19) United States
(12) Patent Application Publication
(10) Pub. No.: US 2010/0003680 A1
(43) Pub. Date: Jan. 7, 2010

(54) Method For Determining The Methylation Rate of a Nucleic Acid

(76) Inventors: Joern Lewin, Berlin (DE); Ralf Lesche, Berlin (DE); Matthias Schuster, Berlin (DE); Dimo Dietrich, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 12/309,555

(22) PCT Filed: Jul. 18, 2006
(86) PCT No.: PCT/EP2007/006064

§ 371 Date: Jan. 21, 2009

(51) Int. Cl. C12Q 1/68; C07H 21/00

(52) U.S. Cl. 435/6; 536/23.1

(57) ABSTRACT

The invention relates to a method for quantitatively determining the methylation rate of a nucleic acid through sequencing. According to the invention, the method comprises at least the following steps: a) treating the nucleic acid with a chemical reagent or an enzyme containing solution, whereby the base pairing behavior of methylated cytosine bases and/or unmethylated cytosine bases of the nucleic acid are altered such that methylated cytosine bases become distinguishable from unmethylated cytosine bases, and b) introducing into the nucleic acid at least one base for generating a sequencing signal to be used as a reference signal for normalization, and c) sequencing the nucleic acid, whereby a signal from each cytosine base of the nucleic acid, or a signal from each guanine base of the nucleic acid and a reference signal from the at least on introduced base is obtained, and d) normalizing the signal obtained from each cytosine base of the nucleic acid, or the signal obtained from each guanine base of the nucleic acid to the reference signal from the at least one introduced base.

The invention relates to a method for quantitative sequencing of methylated DNA according to claim 1, to an oligonucleotide according to claim 25, to kits for the realization of these methods according to claims 26 and 26, and to the use of these methods and these kits according to claims 28, 29, and 30.

Throughout this application, various publications are cited. The disclosure of these publications is hereby incorporated by reference in its entirety into this application to describe more fully the state of the art to which this invention pertains.

Gene regulation has been correlated with methylation of a gene or genome. Certain cell types consistently display specific methylation patterns, and this has been shown for a number of different cell types (Adorjan et al. (2002) Tumor class prediction and discovery by micro array-based DNA methylation analysis. Nucleic Acids Res 30(5) e21).

In vertebrates, DNA is methylated nearly exclusively at cytosine bases located 5′ to guanine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females.

Differential methylation patterns have great relevance for understanding disease and diagnostic applications. The identification of 5-methylcytosine within a DNA sequence is of importance in order to uncover its role in gene regulation. The position of a 5-methylcytosine cannot be identified by a normal sequencing reaction, since it behaves just as an unmethylated cytosine as per its hybridization preference.

Furthermore, in any standard amplification or sequencing reaction, such as the Sanger sequencing method (Sanger F, Nicklen S, Coulson A R. (1977) DNA sequencing with chain-terminating inhibitors. Proc Natl Acad Sci USA, 74(12): 5463-5467), this relevant epigenetic information will be lost.

Several methods are known to solve this problem. Generally, genomic DNA is treated with a chemical or enzyme leading to a conversion of the cytosine bases, which consequently allows one to distinguish between methylated and unmethylated cytosine. The most common methods are a) the use of methylation-sensitive restriction enzymes capable of differentiating between methylated and unmethylated DNA and b) treatment with bisulfite. The use of methylation-sensitive restriction enzymes, however, is limited due to the selectivity of the restriction enzyme towards a specific recognition sequence.

In contrast, bisulfite specifically reacts with unmethylated cytosine regardless of the surrounding sequence. Upon subsequent alkaline hydrolysis, the unmethylated cytosine is converted to uracil, while 5-methylcytosine remains unmodified during this treatment (Shapiro et al. (1970) Nature 227: 1047)). Therefore, it is currently the most favored method of use for analyzing DNA for the presence of 5-methylcytosine. Uracil exhibits the same base pairing behavior as thymine; that is, it hybridizes with adenine. 5-methylcytosine does not change its chemical properties under this treatment and therefore still hybridizes with guanine. Consequently, under bisulfite treatment, the original DNA is converted in such a manner that 5-methylcytosine can now be detected as cytosine whereas those cytosines that were unmethylated in the original DNA can now be detected as thymine. Due to this, 5-methylcytosine, which originally could not be distinguished from cytosine by its hybridization behavior, can now be differentiated from cytosine using conventional molecular biological techniques, such as amplification and hybridization or sequencing, which are based on base pairing, and can now be fully exploited. Comparing the sequences of the DNA with and without bisulfite treatment allows easy identification of those cytosines that have been methylated.

An overview of the further known methods of detecting 5-methylcytosine may be gathered from Fraga F M and Esteller M, Biotechniques (2002) 33(3): 632, 634, 636-649.

As the use of methylation-specific enzymes is dependent on the presence of restriction sites, most methods are based on a bisulfite treatment that is conducted before a detection or amplifying step (for review: DE 100 29 915 A1, page 2, lines 35-46 or the according translated U.S. application Ser. No. 10/311,661; see also WO 2004/067545).

The term ‘bisulfite treatment’ is meant to comprise treatment with a bisulfite, a disulfite or a hydrogensulfite solution. As known to the expert skilled in the art and according to the invention, the term “bisulfite” is used interchangeably for “hydrogensulfite”.

Several laboratory protocols are known in the art, all of which comprise of the following steps: The genomic DNA is isolated, denatured, converted several hours by a concentrated bisulfite solution, and finally desulfonated and desalted (e.g.: Frommer et al. (1992) A genomic sequencing protocol that yields a positive display of 5-methylcytosine residues in individual DNA strands. Proc Natl Acad Sci U.S.A.; 89(5): 1827-1831).

Subsequent to a bisulfite treatment, usually short, specific fragments of a known gene are amplified and either completely sequenced (Olek A, Walter J. (1997) The pre-implantation ontogeny of the H19 methylation imprint. Nat. Genet. 3: 275-6) or individual cytosine positions are detected by a primer extension reaction (Gonzalgo M L and Jones P A (1997) Rapid quantitation of methylation differences at specific sites using methylation-sensitive single nucleotide primer extension (Ms-SNuPE). Nucleic Acids Res 25: 2529-2531, WO 95/00669) or by enzymatic digestion (Xiong Z, Laird P W. (1997) COBRA: a sensitive and quantitative DNA methylation assay. Nucleic Acids Res 25: 2535-2534).

The treatment with bisulfite (or similar chemical agents or enzymes) with the effect of altering the base pairing behavior of one type of cytosine specifically, either the methylated or the unmethylated, thereby introducing different hybridization properties, makes the treated DNA more applicable to the conventional methods of molecular biology, especially the polymerase-based amplification methods, such as the sequencing method based on Sanger F, Nicklen S, Coulson A R. (1977) DNA sequencing with chain-terminating inhibitors. Proc Natl Acad Sci USA, 74(12): 5463-5467).

A quantification of the degree of methylation is necessary for different applications, e.g., for classifications of tumors, for prognostic information or for the prediction of drug effects. Different methods are known for the quantification of the degree of methylation, e.g. by Ms-SNuPE, by hybridizations on microarrays, by hybridization assays in solution or with by bisulfite sequencing (for review: Fraga and Estella (2002), Biotechniques 33(3): 632, 634, 636-649.). A powerful quantification tool based on real time PCR detection is the so called “QM-Assay” described in PCT/EP2005/003793.

In order to characterize the methylation patterns of different tissue types genome wide, methods are required that can detect methylation patterns by automated high throughput technologies in a reliable manner.

Typically, a tissue sample contains a mixture of different cells. Therefore, a proper description of methylation at a certain CpG site requires quantification of the proportion of the methylated templates at the investigated CpG. This proportion is herein referred to as the “methylation rate” of the CpG. After bisulfite conversion and amplification, e.g. by PCR, the methylation rate at a CpG can be determined by assessing the proportion of remaining cytosine bases relative to the number of thymine bases. This can be done, e.g. by hybridization to oligomer probes on DNA chips (Adorjan et al., (2002) Tumour class prediction and discovery by microarray-based DNA methylation analysis. Nucleic Acids Res., 30: e21) or by DNA sequencing (Frommer et al., (1992) A genomic sequencing protocol that yields a positive display of 5-methylcytosine residues in individual DNA strands. Proc Natl Acad Sci USA, 89: 1827-1831). Commonly used sequencing methods include the sequencing of a representative number of sub-clones of the PCR product or direct PCR sequencing by running independent sequencing reactions for cytosine and thymine using the same dye in different lanes of a sequencing gel (Paul and Clark, (1996) Cytosine methylation: quantitation by automated genomic sequencing and GENESCAN analysis. BioTechniques, 21: 126133). These sequencing methods, however, are expensive and labor intensive.

Alternatively, direct PCR sequencing on standard sequencing machines is used to achieve the required throughput in a more cost effective way. This technology produces four-dye electropherogram data. The possibility to use such data for quantitative analyses of base compositions within pooled DNA was recently demonstrated for one single nucleotide polymorphism (SNP) (Qiu et al., (2003) Quantification of single nucleotide polymorphisms by automated DNA sequencing. Biochem. Biophys. Res. Commun., 309: 331-338).

In order to measure methylation according to Lewin et al. 2004, genomic DNA is bisulfite treated and applied to sequencing, wherein also a multiple dye electropherogram is necessary (Lewin J, et al., (2004) Quantitative DNA methylation analysis based on four-dye trace data form direct sequencing of PCR amplificates. Bioinformatics 20 (17): 3005-3012).

The sense strand of a native nucleic acid is the strand that serves as a template for RNA transcription. The anti-sense strand of a native nucleic acid is the strand of the nucleic acid which is complementary to the sense strand and with which it forms a duplex nucleic acid. After bisulfite treatment, these two strands are no longer complementary to each other. Both of them contain low quantities of cytosine bases, since all unmethylated cytosines have been converted into uracils. For this reason, these two strands are referred to as “G-rich strands” (guanosines are more abundant in comparison to cytidines). In contrast to native DNA, these two strands are herein also called “bisulfite sense strands”. Both of these bisulfite sense strands may act as a template DNA for analyzing the same CpG sites. Accordingly, the strands which are generated during an amplification reaction, and which are complementary to these bisulfite sense strands, are herein referred to as “bisulfite anti-sense strands”. Since bisulfite anti-sense strands are complementary to the bisulfite sense strands, they contain low quantities of guanosines but high quantities of cytidines and are therefore also called “C-rich strands”.

Quantitative analysis by direct sequencing of the amplification, e.g. PCR products from bisulfite-treated DNA implicates several novel challenges:

1) Signal quality is generally poor compared to signals stemming from genomic sequencing.

2) Bisulfite treatment leads to the degradation of the treated nucleic acid. This is especially problematic when using genomic DNA that is already degraded. Body fluids as wells as archived sources, such as formaline-fixed and paraffin embedded tissues are well known to contain degraded DNA. Because of the degradation by bisulfite treatment and, if the case may be, of the applied genomic DNA, only short fragments of the nucleic acid can be amplified. These short fragments are difficult to sequence because the signal resolution at the beginning of a sequencing read is of poor quality rendering the base caller unable to identify the bases accurately.

3) Cytosine signals (in the bisulfite sense strand) and guanosine signals (in the bisulfite anti-sense strand) are overscaled due to base caller artifacts. The so-called “base caller” is a program of the sequencing machine supplied by the manufacturer, e.g. Applied Biosystems “.abi” files or the well-described “.scf” files (Dear and Staden, (1992) A standard file format for data from DNA sequencing instruments. DNA Seq., 3: 107-110). Since cytosine signals are scarce in bisulfite-treated DNA, the remaining cytosine signals are over-scaled by the base caller program, leading to too great a signal for the detected cytosine bases. Therefore, a reliable determination of the methylation rate is not feasible.

4) In combination with the over-scaled signals, incomplete bisulfite conversion, which is a general problem of all bisulfite-based methylation detection methods, influences signal proportions in the sequencing trace significantly.

Bisulfite genomic DNA sequencing offers a continuous readout of the entire, detailed, base-by-base methylation map of a genomic DNA sequence (Frommer et al. (1992) Genomic sequencing protocol that yields a positive display of 5-methylcytosine residues in individual DNA strands. Proc Natl Acad Sci USA 89: 1827-1831; R. Feil et al. (1994) Methylation Analysis on Individual Chromosomes—Improved Protocol for Bisulfite Genomic Sequencing. Nucleic Acids Res 22: 695696). The technique also relies on initial bisulfite modification of DNA and, as a final step, direct cycle sequencing of the resulting PCR-amplified sequence. PCR primers are designed external to potential methylation sites. However, because of the bisulfite conversion of unmethylated C to U in the template, there is a paucity of C (in the bisulfite sense strand) or G (in the bisulfite anti-sense strand) nucleotides in the PCR product. Thus, there is a low GC content, rendering direct cycle sequencing extremely challenging and often uninterpretable.

To overcome these problems, conventional bisulfite sequencing commonly requires the cloning of the PCR product for two reasons: First, the incorporation into the plasmid vector allows low GC content to be compensated for by the external plasmid sequence. Second, this approach provides precise methylation patterns of individual DNA molecules, overcoming tissue heterogeneity issues affecting methylation patterns at individual CpG sites. However, this requirement renders conventional bisulfite genomic DNA sequencing time consuming and labor intensive, and it precludes large-scale surveillance studies.

A tag-modified bisulfite genomic DNA sequencing method (tBGS) was reported by Han et al. 2006 (Han W et al. (2006) DNA methylation mapping by tag-modified bisulfite genomic sequencing, Anal Biochem 355: 50-61), which yields a 5′ and 3′ GC-tagged PCR product that enhances the GC content to allow direct cycle sequencing. According to this technology, however, methylation is analysed by comparing the signal of cytosine with the signal of thymin at a possible methylated position. Therefore the analysis requires the detection and analysis of all four trace-signals. In addition, the method does not allow for quantitative analysis of methylation rates.

Therefore, the problem underlying the present invention was to provide a method for sequencing of methylated DNA that could be performed in an easy and reliable manner to determine the methylation rate of a nucleic acid. Furthermore, a kit was to be supplied with which the method according to the present invention could be realized.

Surprisingly, the inventors were able to solve this problem by inventing the present method. The central idea of the invention is to provide an artificially introduced base into the nucleic acid to be analyzed, that provides for a reference signal when the nucleic acid to be analyzed is sequenced. This reference signal can then be used in a normalization procedure for all the cytosine (or guanine) signals stemming from the nucleic acid to be analyzed that were obtained in the sequencing reaction.

This newly developed data analysis method allows the use of established high-throughput sequencing technology for methylation studies using only one sequencing dye. It is furthermore useful for sequencing a target DNA molecule to be analyzed in a pool of different DNA molecules.

### DESCRIPTION OF THE INVENTION

Disclosed is a method for determining the methylation rate of a nucleic acid through sequencing.

According to the method of the present invention, at least the following four steps will be performed:
First, a template DNA that is to be analyzed with respect to its methylation rate is treated with at least one chemical reagent or with at least one solution containing at least one enzyme, whereby the base pairing behavior of methylated cytosine bases and/or unmethylated cytosine bases of the nucleic acid are altered such that methylated cytosine bases become distinguishable from unmethylated cytosine bases in terms of their hybridization properties, i.e. base pairing properties.
Secondly, into the nucleic acid from the first step, in which all (formerly) methylated cytosine bases become distinguishable from all (formerly) unmethylated cytosine bases in terms of their base pairing properties, at least one base for generating a reference signal in a sequencing reaction is introduced. This reference signal stemming from the introduced base will be used for normalization in step four.
Thirdly, the nucleic acid with the introduced at least one reference base is sequenced, a signal from each cytosine base of the nucleic acid, or a signal from each guanine base of the nucleic acid, and a reference signal from the at least on introduced base is obtained.
Fourth, for the bisulfite sense strand (G-rich strand), the signal obtained from each cytosine base of the nucleic acid is normalized to the reference signal from the at least one introduced base. For the bisulfite anti-sense strand (G-rich strand), the signal obtained from each guanine base of the nucleic acid is normalized to the reference signal from the at least one introduced base. In other words, a signal stemming from sequencing the introduced base is used as a reference signal for normalization of all the signals stemming from the cytosine (or guanine) bases of the nucleic acid to be analyzed.

The first two steps mentioned above can be performed also in reverse order, that is first introducing the reference signal into the nucleic acid to be analyzed and then treating, i.e. converting the nucleic acid such that methylated cytosine bases are distinguishable form unmethylated cytosine bases, if the introduced reference base will not be converted itself during treatment. This is the case in particular for guanine and for certain base analogs, which do not occur naturally.

The method according to the present invention is useful for analyzing the methylation rate of nucleic acid samples, i.e. determining to what percentage a certain CpG position of a nucleic acid population has been methylated. Advantageously, the method can be performed using only one sequencing dye, namely for the cytosine (or guanine) signal trace, since the introduced base can also detected using the same sequencing dye.

The method is further advantageous, because it can be successfully used on short nucleic acid molecules, particularly those nucleic acid molecules that have been partially degraded. Such degraded nucleic acids can be found in body fluid or in formalin-fixed tissue samples.

The method of the invention is further advantageous with respect of the prior art, because it is able to directly interpret raw sequencing data without any pre-processing for example by means of a base caller algorithm. Pre-processing algorithm are applied to most sequencings by default. This is in particular done in order to be able to compare the signal derived from one base with the signals derived from the other bases. A preprocessing is necessary wherein the signals derived for at least two bases are regarded. However, preprocessing is not necessary according to the invention, because only the signals of either cytosine or guanine are considered.

In contrary thereto, the methods of the state of the art are based on a comparison of at the least the cytosine signal with the thymine signal. According to the method of Han et al. (supra) a cytosine base or guanin base is introduced, but however this is only been done in order to achieve an accurate preprocessing by an preprocessing algorithm (base caller). However, methylation is determined from the signals of cytosine and thymine. Also according to Lewin et al. (supra) methylation is determined from the cytosine and thymine signal. In particular, the normalization value according to Lewin et al. is determined by combining the signals of cytosine and thymine at CpG positions. Though Lewin et al. discloses also an embodiment which is based on the consideration of only one base i.e. the signals derived from thymine, this embodiment allows only the direct detection of non-methylation. Thereby a normalization is carried out against the signal of thymine at positions wherein thymine occurs in genomic DNA or wherein cytosines outside of CpG positions are converted to thymine. However, this embodiment of Lewin et al. has the disadvantage that methylation is only determinable indirectly.

In addition, the method of the invention has the advantage that normalization occurs with a reference signal of the same base (i.e. the introduced base(s)) as the analyzed signals (i.e. signals of cytosines or guanins). This excludes that signal noise leads an adulteration of results.

The normalized height of the signals (peak maxima) stemming from the cytosine (or in the other case guanine) bases of the nucleic acid to be analyzed, directly represent the methylation rate of the analyzed nucleic acid. The normalized signals at each position at which a cytosine (or guanine) signal was detected can also be integrated to obtain a normalized area of the signal. The said normalized area is a more robust value of the methylation rate than one would get when determining the peak heights alone because it is based on a plurality of measured values. In either case, the methylation rate of a nucleic acid can be determined quantitatively through sequencing. In a preferred embodiment, the height of signals (peak maxima) are used for normalization. In a particular preferred embodiment, the area of signals are used for normalization.

In a preferred embodiment of the present invention, the methylation rate of an analyzed nucleic acid thereby obtained can be further corrected by comparing the normalized signals (areas or heights) with normalized signals (areas or heights, respectively) of analyzed standard nucleic acids with known methylation rates (calibration). This way, one can determine the percentage of methylation at a defined position, yielding the methylation index.

It is preferred that the at least one introduced base is at least one artificial base analog, and/or at least one cytosine base, and/or at least one guanine base.

A base analog is a base that is not naturally occurring, and that differs from the naturally occurring A, C, G, and T. Such base analogs are well known in the art and for example but not limited to described in Sismour and Brenner 2005, Johnson et al. 2004, Yang et al. 2006 or Brenner and Sismour 2005 (Sismour A M and Benner S A (2005) The use of thymidine analogs to improve the replication of an extra DNA base pair: a synthetic biological system. Nucleic Acids Res. 33(17): 5640-5646; Johnson S C, Sherrill C B, Marshall D J, Moser M J, Prudent J R. (2004) A third base pair for the polymerase chain reaction: inserting isoC and isoG. Nucleic Acids Res. 32(6): 1937-1941; Yang Z, Hutter D, Sheng P, Sismour A M, Benner S A. Artificially expanded genetic information system: a new base pair with an alternative hydrogen bonding pattern. Nucleic Acids Res. 006; 34(21):6095-101; Benner S A, Sismour A M. Synthetic biology. Nat Rev Genet. 2005 July; 6(7):533-43). The base pairs of base analogs which are complementary to each other exhibit a different hybridization behavior than A/T and G/C. In a preferred embodiment the base analog is isocytosine, isoguanine, 2-thiothymidine, 6-amino-5-nitro-3-(1′-beta-D-2′-deoxyribofuranosyl)-2(1H)-pyridone (dZ), or 2-amino-8-(1′-beta-D-2′-deoxyribofuranosyl)-imidazo[1,2-a]-1,3,5-triazin-4(8H)-one (dP).

As described above, this method can be used on a nucleic acid that has been treated with a chemical reagent or with a solution containing at least one enzyme, whereby the base pairing behavior of methylated cytosine bases and/or unmethylated cytosine bases of the nucleic acid are altered such that methylated cytosine bases become distinguishable from unmethylated cytosine bases in terms of their hybridization properties. Furthermore, this method can also be applied on a nucleic acid which is reverse complementary in sequence to the treated nucleic acid and was generated by an amplification reaction, e.g. by a polymerase chain reaction (PCR). It can be necessary, however, to introduce a different at least one base for each particular nucleic acid. For example, if bisulfite was used as a chemical reagent, as explained below, a cytosine is to be introduced into the G-rich strand (bisulfite sense strand) according to the present method and a guanine is to be introduced into the C-rich strand (bisulfite anti-sense strand). Base analogs can be used regardless of the base composition of the nucleic acid.

When sequencing the bisulfite sense strand (G-rich strand), a signal from each cytosine base of the nucleic acid and a reference signal from the at least one introduced base is obtained. When sequencing the bisulfite anti-sense strand (C-rich strand; generated by amplification of the bisulfite sense strand/G-rich strand), a signal from each guanine base of the nucleic acid and a reference signal from the at least one introduced base is obtained.

In a preferred embodiment of the present invention, the at least one introduced base is introduced into the nucleic acid as part of an at least one nucleotide. Introduction into the nucleic acid is easiest if the at least one nucleotide is itself part of an at least one oligonucleotide, which then comprises at least one base analog, and/or at least one cytosine base, and/or at least one guanine base to generate a reference signal in the sequencing procedure.

Although it is generally sufficient if the at least on oligonucleotide comprises only one reference base, best results are achieved if the at least one oligonucleotide comprises two to four, most preferably three introduced bases for generating a sequencing signal to be used as a reference signal. But of course it is also possible to use five, sex, seven, eight, nine, ten or more than ten reference bases. In a preferred embodiment, the at least one oligonucleotide comprises three cytosine bases or base analogs for sequencing the bisulfite sense strand or three guanine bases or base analogs for sequencing the bisulfite anti-sense strand. A greater number of reference signals allows for choosing an appropriate signal to be used in the normalization calculation. Alternatively, a mean reference value can be calculated from all reference signals stemming from the at least one oligonucleotide, and this mean reference value can then be used to normalize the signals stemming from the nucleic acid to be analyzed.

Preferably, the nucleic acid to be analyzed is enzymatically converted. For example but not limited to by means of cytidin deaminases. These enzymes convert unmethylated cytosine faster than methylated cytosine (Bransteitter et al.: Activation-induced cy-tidine deaminase deaminates deoxycytidine on single-stranded DNA but requires the action of RNase. Proc Natl Acad Sci USA. 2003 Apr. 1; 100(7):4102-7; or WO 2005/005660).

Preferably, the nucleic acid to be analyzed is incubated with a bisulfite containing solution, whereby unmethylated cytosine bases of the nucleic acid are converted into sulfon-uracil bases or uracil bases while 5-methylcytosine bases remain unchanged, yielding a G-rich nucleic acid. A conversion into uracil bases occurs, wherein the bisulfite conversion comprises the desulfonation of the treated nucleic acid by increasing the pH. A conversion into sulfon-uracil bases occurs, wherein the bisulfite conversion is free of a desulfonation step. Desulfonation is then carried out subsequently by increasing the temperature for example before or in the sequencing reaction (WO 2006/040187).

This is preferably achieved by means of treatment with a bisulfite reagent, will later be described with reference to FIG. 1. The term “bisulfite reagent” refers to a reagent comprising bisulfite, disulfite, hydrogen sulfite or combinations thereof, useful as disclosed herein to distinguish between methylated and unmethylated CpG dinucleotide sequences. Methods of said treatment are known in the art (e.g. PCT/EP 2004/011715).

It is preferred that the bisulfite treatment is conducted in the presence of denaturing solvents, such as, but not limited to, n-alkylenglycol, particularly diethylene glycol dimethyl ether (DME), or in the presence of dioxane or dioxane derivatives. In a preferred embodiment, the denaturing solvents are used in concentrations between 1% and 35% (v/v). It is also preferred that the bisulfite reaction is carried out in the presence of scavengers such as, but not limited to, chromane derivatives, e.g. 6-hydroxy-2,5,7,8-tetramethylchromane 2-carboxylic acid or trihydroxybenzoe acid and derivates thereof, e.g. Gallic acid (see: PCT/EP2004/011715). The bisulfite conversion is preferably carried out at a reaction temperature between 30° C. and 70° C., whereby the temperature is increased to over 85° C. for short periods of times during the reaction (see: PCT/EP2004/011715). The bisulfite-treated DNA is preferably purified prior to the quantification. This may be conducted by any means known in the art, such as, but not limited to, ultrafiltration, preferably carried out by means of Microcon™ columns (manufactured by Millipore™). The purification is carried out according to a modified manufacturer's protocol (see: PCT/EP2004/011715).

It is also preferred that the bisulfite reaction is carried out in the presence of scavengers such as but not limited to chromane derivatives, e.g., 6-hydroxy-2,5,7,8-tetramethylchromane 2-carboxylic acid or trihydroxybenzoe acid and derivates thereof, e.g. Gallic acid (see: PCT/EP2004/011715 which is incorporated by reference in its entirety). The bisulfite conversion is preferably carried out at a reaction temperature between 30° C. and 70° C., whereby the temperature is increased to over 85° C. for short periods of times during the reaction (see: PCT/EP2004/011715 which is incorporated by reference in its entirety). The bisulfite-treated DNA is preferably purified priori to the quantification. This may be conducted by any means known in the art, such as but not limited to ultrafiltration, preferably carried out by means of Microcon™ columns (manufactured by Millipore™). The purification is carried out according to a modified manufacturer's protocol (see: PCT/EP2004/011715).

Instead of a chemical conversion, it is furthermore possible to conduct the conversion enzymatically, e.g. by use of methylation-specific cytidine deaminases (German Patent DE 103 31 107 B; PCT/EP2004/007052).

The at least one base can be conveniently introduced into the nucleic acid through two possible ways, namely through an amplification reaction or a ligation reaction.

First, embodiments of the invention that make use of an amplification reaction will be described.

In a preferred embodiment of the method, the bisulfite treated nucleic acid is amplified using at least one oligonucleotide that comprises at least one cytosine base and/or at least one base analog, whereby the at least one oligonucleotide is incorporated into a G-rich nucleic acid. The at least one oligonucleotide may further comprises a sequencing domain for hybridization of a sequencing primer, wherein the sequencing domain is located on the 5′ side of the at least one cytosine base and/or of the at least one base analog, enabling the hybridization of a sequencing primer to the amplification product.

For generating the reverse complementary strand (bisulfite anti-sense or C-rich strand) to the treated nucleic acid, the bisulfite treated nucleic acid is amplified using at least one oligonucleotide that comprises at least one guanine base and/or at least one base analog, whereby the at least one oligonucleotide is incorporated into a C-rich nucleic acid. To simplify sequencing of the amplification product, the at least one oligonucleotide can further comprise a sequencing domain for hybridization of a sequencing primer, with the sequencing domain located on the 5′ side of the at least one guanine base and/or of the at least one base analog.

It is particularly preferred that the at least one oligonucleotide serves as a primer for the amplification reaction.

Two ways are possible in designing the at least one oligonucleotide that serves as a primer in the amplification reaction: First, the at least one cytosine or guanine base can be located at the 5′ end of the oligonucleotide, and on the 3′ side of that region a nucleotide sequence is located which hybridizes with the nucleic acid to be analyzed. This way, the oligonucleotide has a sequence part at the 5′ tail end that comprises at least one cytosine and/or at least one guanine base and/or at least one base analog, which will generate a sequencing reference signal, and a 3′ end that hybridizes with the nucleic acid to be analyzed and primes the nucleic acid amplification reaction.

It is preferred that the 5′ end of the oligonucleotide that serves as a primer in the amplification reaction comprises an asymmetric sequence. This has the advantage that the occurrence of primer dimers is avoided. In a particular preferred embodiment, the said 5′ end comprises the sequence “CC”, “CCC”, “CCCC”, “CCCCC”, or more than five cytosines in a row, or combinations of said sequences. In a particular preferred embodiment, the said 5′ end comprises the sequence “GG”, “GGG”, “GGGG”, “GGGGG”, or more than five guanins in a row, or combinations of said sequences. In a particular preferred embodiment, the said 5′ end comprises the sequence of two, three, four, five or more base analogs in a row, or combinations of said sequences. In addition, it is particularly preferred that the said sequence comprises, contains or is the sequence ACTCC (for the bisulfite sense strand) or AGGTG (for the bisulfite anti-sense strand). It is particularly also preferred that the said sequence comprises, contains or is the sequence CGTCGTCG.

Secondly, the at least one cytosine base, guanine base, or base analog which generates the sequencing reference signal can be embedded within a portion of the at least one oligonucleotide that hybridizes (is complementary) to the strand of the nucleic acid to be analyzed. In this case, the at least one cytosine or guanine base can hybridize with a guanine or (methylated) cytosine base of the opposite strand, respectively, or can mismatch with a base on the opposite strand. When using a base analog as an introduced base, a non Watson-Crick base pairing will occur. At least one or preferably at least two nucleotides should be located on the 5′ and 3′ side of the corresponding nucleotide that generates a reference signal during the sequencing reaction to allow for an enzyme to bind efficiently and catalyze an amplification reaction.

In a preferred embodiment of the invention, the at least one sequence part that hybridizes with a sequence of the nucleic acid to be amplified of the at least one oligonucleotide has a length of between 10 nucleotides (nt) to 40 nt, preferably between 15 nt to 30 nt, and more preferably between 18 nt to 25 nt.

In a preferred embodiment of the invention that makes use of the at least one oligonucleotide as a primer for an amplification reaction, the primer that comprises the at least one guanine base or the at least one cytosine base or the at least one base analog further comprises a sequencing domain. This sequencing domain will allow a sequencing primer to hybridize and allow sequencing of the amplified nucleic acid. For this purpose, the sequencing domain is located on the 5′ side of the at least one cytosine or guanine base, so that the reference signals used for normalization are also sequenced. This embodiment has the advantage that the normalization signal is located at the beginning of a sequencing read, independent of the sequence of the analyzed nucleic acid and is therefore easily automatically detectable.

Base analogs can be advantageously used within an at least one oligonucleotide used as a primer for an amplification reaction. Specifically, a primer can be used that contains e.g. at least one iso-cytosine (isoC) or at least one iso-guanine (isoG). During the cycle sequencing reaction, an additional dye-labeled dideoxyribonucleoside triphosphates (ddNTP) is added to the sequencing reaction mixture. This additional ddNTP can be, e.g. ddisoCTP (in case of an iso-guanine containing primer in the amplification reaction) or ddisoGTP (in case of an iso-cytosine containing primer in the amplification reaction) and is labeled with the same sequencing dye as the nucleotide (dNTP) generating the signals for the guanine or cytosine bases of the nucleic acid.

As a result of this modification, the normalization signal is generated by a not naturally occurring base analog. This has two advantages, namely that first, both primers (for amplifying the sense and the anti-sense strand, respectively) may contain an identical tag and that secondly, this tag does not interfere with any site within the bisulfite-treated genome during the amplification reaction. This increases the comparability of the sequencing results of the sense and anti-sense strand and also increases the specificity of the amplification of the nucleic acid.

In a preferred embodiment, which will be further described below with reference to FIG. 17, an enzymatic amplification reaction is performed using a first oligonucleotide comprising at least one cytosine base (for amplifying the bisulfite sense strand), and a second oligonucleotide comprising at least one guanine base (for amplifying the bisulfite anti-sense strand). At least one of these two primers, that is the first or the second oligonucleotide, further comprises a sequencing domain. This sequencing domain will allow a sequencing primer to hybridize and allow sequencing of the amplified nucleic acid. For this purpose, the sequencing domain is located on the 5′ side of the at least one cytosine or guanine base, so that the reference signals used for normalization are also sequenced. This embodiment has advantages for x-axis normalization, as will be discussed below.

The oligonucleotides used as primers will be chosen such that they amplify a fragment of interest. It is particularly preferred that these oligonucleotides are designed to amplify a nucleic acid fragment of a template nucleic acid sample by means of a polymerase reaction, in particular a polymerase chain reaction (PCR), as known in the art. The oligonucleotides are therefore designed to anneal to the template nucleic acids, to form a double strand, following the Watson-Crick base pairing rules (with the exception of introducing mismatching bases into the at least one oligonucleotide, as mentioned above). The length of the two oligonucleotides used in one amplification reaction will be selected such that they anneal at approximately the same temperature.

Alternatively, the PCR can also be performed using oligonucleotides (primers) without at least one introduced base. In that case, an appropriate oligonucleotide providing a reference sequencing signal can be introduced into the nucleic acid after the amplification reaction, using ligation.

The following embodiments of the invention make use of a ligation reaction to introduce the at least one base into the nucleic acid.

According to one such embodiment, wherein the at least one oligonucleotide is introduced into the nucleic acid through ligation, the at least one oligonucleotide comprises at least one cytosine base and/or at least one base analog for sequencing a G-rich strand of the nucleic acid, or at least one guanine base and/or at least one base analog for sequencing a C-rich strand of the nucleic acid.

In order to make ligation of the at least one oligonucleotide more efficient, the at least one oligonucleotide can be hybridized with a reverse complementary oligonucleotide to form a double-stranded nucleic acid. This double-stranded nucleic acid is then ligated to the nucleic acid which can then be analyzed through sequencing. The ligation step can be performed such that both the oligonucleotide and the nucleic acid to be analyzed are blunt ended. However, it is preferred that ligation is performed between molecules with sticky ends. Ways of performing the ligation reaction are known to a person skilled in the art.

In a preferred embodiment of this alternative method of the invention, the at least one oligonucleotide comprises a sequencing domain for hybridization of a sequencing primer, wherein the sequencing domain is located on the 5′ side of the at least one cytosine base and/or at least one base analog or guanine base and/or at least one base analog.

This sequencing domain will allow a sequencing primer to hybridize to the at least one oligonucleotide and allow sequencing of the amplified nucleic acid. For this purpose, the sequencing domain of the at least one oligonucleotide is located on the 5′ side of the at least one cytosine or guanine base, so that the at least one reference signal used for normalization is sequenced together with the nucleic acid.

It is preferred that the sequencing reaction of the amplified nucleic acid is performed using cycle sequencing, as known in the art.

In a preferred embodiment of the invention, the enzyme-based amplification reaction is started by heat-activation, that is, by a brief incubation at an increased temperature, which activates the enzymatic activity. For this purpose, a heat stable enzyme is preferred.

In case the enzyme-based amplification reaction is a polymerase-based amplification reaction, in particular a polymerase chain reaction (PCR), it is preferred that the enzyme is a heat stable polymerase. However, it is also possible to apply other enzymatic amplification reactions known to the person skilled in the art, including, but not limited to, ligase-mediated amplifications (e.g. Ligase-Chain Reaction) or amplifications based on transcription (e.g. NASBA™, 3SR™, TMA™).

The sequencing reaction is preferably performed using one kind of labeled dideoxyribonucleoside triphosphates that forms base pairs with either the cytosine bases of the nucleic acid and the introduced base, or the guanine bases of the nucleic acid and the introduced base. This way, the method can be performed using only one sequencing dye and signals from other bases than from the bases of interest (guanine or cytosine) need not be recorded.

After or while sequencing, the at least one signal stemming from the at least one introduced base of the at least one oligonucleotide is identified. If more than one base was introduced into the nucleic acid, an appropriate signal has to be chosen to be used in the normalization calculation. Alternatively, a mean reference value can be calculated from all reference signals, and this mean reference value can then be used to normalize the signals stemming from the nucleic acid to be analyzed.

The sequencing signal applied to the method of the invention, is either unprocessed (raw data) or preprocessed (for example by a base caller algorithm). In a preferred embodiment the sequencing signal is applied to the method of the invention as raw data. This has the advantage that more accurate results are obtained. The known algorithms for preprocessing of sequencing data are all based on certain assumptions about the frequencies of occurrence of the four bases A, T, C, G. A completely correct preprocessing would only be possible if the exact frequency of each of the four bases would already been known prior sequencing. Because this is not the case for methylation analysis by DNA conversion and subsequent sequencing (the frequencies or occurrence of cytosine is the subject matter of the analysis), any methylation analysis, wherein a pre-processing algorithm (e.g. base caller algorithm) is used is error-associated.

However, it is also possible and herewith preferred to apply the pre-processed sequencing signals to the method of the invention. Many times, sequencing is performed as an automated process, wherein a pre-processing is applied by default. Therefore the use of pre-processed sequencing data has the advantage, that the method of the invention can be directly applied without the need to change the sequencing process. This may lead to error-associated results, wherein the assumpted frequencies of base occurance differ from the actual frequencies, in particular in cases wherein the sequencing is performed in order to quantitatively determine methylation. A qualitative determination may be uneffected i.e. correct.

The sequencing signal is usually represented by a time-dependent intensity curve. In a preferred embodiment, the area under the sequencing curve is determined for each signal stemming from a cytosine base or a guanine base of the nucleic acid and for each signal stemming from the at least one introduced base, to yield area values. Normalization is performed for each signal obtained by dividing the area value of each signal stemming from a cytosine base or a guanine base of the nucleic acid by the area value of the signal from the at least one introduced base (or, in case of more than one introduced bases, of the chosen reference signal or of the calculated mean reference signal).

In a preferred embodiment, the height of sequencing curve peaks is determined for each signal stemming from a cytosine base or a guanine base of the nucleic acid and for each signal stemming from the at least one introduced base, to yield height values. Normalization is performed for each signal obtained by dividing the height value of each signal stemming from a cytosine base or a guanine base of the nucleic acid by the height value of the signal from the at least one introduced base (or, in case of more than one introduced bases, of the chosen reference signal or of the calculated mean reference signal).

When sequencing the same nucleic acid several times, differences in the length of the base trace peaks can be observed, i.e. peaks can be narrower or broader with each run. This can be compensated for by the reference signal, if the determination of the peak area is performed using integration of the entire peak. It is, however, also possible to determine the area of a peak by determining the maximum height of a peak together with a certain number of measured values before and after this peak on the time line, e.g. to take 15 measured values before and after the peak value. The area determined this way might be smaller than the actual peak area. Therefore, area values of broader peaks might be smaller than those of narrower peaks. It is thus important to also normalize for the length of the peaks (x-axis normalization). In addition, this x-axis normalization enables an automated analysis, since signals of the methylated cytosine sites occur at the same point on the normalized time line (when analyzing the same nucleic acid sequence).

X-axis normalization can be performed by using two points on the time line that are far apart and normalizing the distance between these two points. When the nucleic acid that was sequenced comprises bases generating reference signals both on the 5′ and 3′ end of the nucleic acid (as described above), then these reference signals can be used for normalization.

Alternatively, the two signals necessary for x-axis normalization can be generated by running two internal size standards parallel with the sequencing reaction on the same gel. Preferably, these standards bear the same dye as the sequencing reaction of the base in question, so that the method according to the invention can be performed using only one dye.

For example, when the nucleic acid that is to be sequenced has 200 nucleotides (nt), the internal standards could be of 220 nt and 250 nt. The internal standards therefore do not interfere with the sequencing reaction but will run more slowly than the sequencing reaction products on a sequencing gel.

Preferably, the nucleic acid is genomic DNA, which may be isolated and/or denatured and therefore present as single strands. It is also possible to use DNA from other sources, such as synthesized DNA that does not stem from a natural source.

The present invention also comprises an oligonucleotide (primer) for amplifying a nucleic acid. Such an oligonucleotide comprises a first sequence part that is reverse complementary to the nucleic acid to be amplified and serves for initiating an amplification reaction, a second sequence part that contains at least one base for generating a sequencing signal to be used for the normalization of sequencing signals stemming from the nucleic acid, and a third sequence part for the hybridization of a sequencing primer. In the oligonucleotide according to the present invention, the second and third sequence part are not identical. Therefore, the at least one base for generating a sequencing signal will be sequenced before the nucleotides of interest of the nucleic acid to be analyzed, which simplifies the determination of the methylation rate.

An oligonucleotide as described above is further described in the description of FIG. 17.

It is preferred that the first sequence part is located at the 3′ end of the oligonucleotide, followed, in 3′ to 5′ direction, by the second and then the third sequence parts. It is furthermore preferred that the at least one base of the second sequence part is a cytosine base, and/or a guanine base, and/or a base analog. For further features of the oligonucleotide, reference is made to the description above regarding the method according to the invention.

The present invention also comprises a kit, in particular a test kit for the realization of the method described above. Such a test kit comprises a chemical reagent, particularly bisulfite, or an enzyme which alters the base pairing behavior of methylated cytosine bases and/or unmethylated cytosine bases of the nucleic acid such that methylated cytosine bases become distinguishable from unmethylated cytosine bases, and at least one oligonucleotide that comprises at least one cytosine base and/or at least one guanine base and/or at least one base analog, and an enzymatic activity for amplifying a nucleic acid using the at least one oligonucleotide as a primer and/or an enzymatic activity for ligating the at least one oligonucleotide to a nucleic acid.

The at least one oligonucleotide of the test kit enables the correct and simple determination of the methylation rate by introducing at least one cytosine base, at least one guanine base, or at least one base analog that generates a sequencing signal which can serve as a reference signal for the normalization of the cytosine (when sequencing the bisulfite sense strand) or guanine signals (when sequencing the bisulfite anti-sense strand) of the nucleic acid that is analyzed. The properties of the at least one oligonucleotide are the same as described above with reference to the method according to the present invention.

The described test kits may further comprise one or more of the additional components, such as:
one or more denaturing reagent and/or solution, for example: dioxane or diethylene glycol dimethylether (DME) or any substance, which is suitable as described in WO 05/038051,
one or more scavenger, for example 6-hydroxy-2,5,7,8-tetramethylchromane 2-carboxylic acid or other scavengers as described in WO 01/98528 or WO 05/038051,
at least one additional primer, which is suitable for the amplification of one or more DNA amplificates,
one or more reaction buffers, which are suitable for a bisulfite treatment and/or a PCR reaction,
nucleotides, which can be dATP, dCTP, dTTG, dUTP and dGTP or any derivative of these nucleotides,
MgCl₂ as a substance or in solution and/or any other magnesium salt, which can be used to carry out a DNA polymerase replication,
DNA polymerase, for example Taq polymerase or any other polymerase with or without proof-reading activity,
any reagent, solution, device and/or instruction which is useful for realization of a method according to the invention.

The method and test kit disclosed here are preferably used for the diagnosis and/or prognosis of adverse events for patients or individuals, whereby diagnosis means diagnose of an adverse event, a predisposition for an adverse event and/or a progression of an adverse event.

These adverse events belong to at least one of the following categories: undesired drug interactions, cancer diseases, CNS malfunctions, damage or disease, symptoms of aggression or behavioral disturbances, clinical, psychological and social consequences of brain damage, psychotic disturbances and personality disorders, dementia and/or associated syndromes, cardiovascular disease, malfunction or damage, malfunction, damage or disease of the gastrointestinal tract, malfunction, damage or disease of the respiratory system, lesion, inflammation, infection, immunity and/or convalescence, malfunction, damage or disease of the body as an abnormality in the development process, malfunction, damage or disease of the skin, of the muscles, of the connective tissue or of the bones, endocrine and metabolic malfunction, damage or disease, headaches or sexual malfunction.

The method and test kits also serve for distinguishing cell types and tissues or for investigating cell differentiation. They also serve for analyzing the response of a patient to a drug treatment.

The method and test kit of the invention can also be used to determine the DNA methylation rate in that positions are methylated or non-methylated compared to normal conditions if a single defined disease exists. In a particular preferred manner they can serve for identifying an indication-specific target, wherein a template nucleic acid is treated according to the method of the present invention, and wherein an indication-specific target is defined as differences in the DNA methylation rate of a DNA derived from a diseased tissue in comparison to a DNA derived from a healthy tissue. The tissue samples can originate from a patient with the single defined disease and from a healthy individual. They can also originate from one patient with the single defined disease diseased only, in which case DNA from the pathological tissue will be compared to DNA from healthy tissue that was obtained from adjacent to the sick tissue of the patient (so-called adjacent analogous normal tissue).

In other words, DNA stemming from a healthy individual and an individual with a single defined disease will be analyzed with respect to its methylation rate at particular CpG sites. The results are then compared to each other with the goal of identifying CpG positions in genomic DNA that allow for the diagnosis of the single defined disease in a patient and/or that allow for the prediction of likelihood of an individual becoming ill with the single defined disease and/or that allow for the prediction of likelihood of an individual surviving with the single defined disease.

In a particular preferred manner, the method and test kit of the invention can serve for identifying an indication-specific target, wherein a template nucleic acid is treated according to the method of the present invention, and wherein an indication-specific target is defined as differences in the DNA methylation rate of a DNA derived from a diseased tissue in comparison to a DNA derived from a healthy tissue. These tissue samples can originate from diseased or healthy patients or from diseased or healthy adjacent tissue of the same patient.

The sample nucleic acid can be obtained from serum or other body fluids of an individual. They can, in particular, be obtained from cell lines, tissue embedded in paraffin, such as tissue from eyes, intestine, kidneys, brain, heart, prostate, lungs, breast or liver, histological slides, body fluids and all possible combinations thereof.

The term body fluids is meant to comprise fluids such as whole blood, blood plasma, blood serum, urine, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile, stool and cerebrospinal fluid. It is especially preferred that said body fluids are whole blood, blood plasma, blood serum, urine, stool, ejaculate, bronchial lavage, vaginal fluid and nipple aspirate fluid.

The present invention can furthermore be used to determine methylation patterns of cells and tissues, both healthy and sick.

In a preferred embodiment, the nucleic acid according to the invention is genomic DNA.

Subject matter of the invention is a method for determining the methylation rate of a nucleic acid through sequencing, comprising the steps of:
treating the nucleic acid with a chemical reagent or an enzyme containing solution, whereby the base pairing behavior of methylated cytosine bases and/or unmethylated cytosine bases of the nucleic acid are altered such that methylated cytosine bases become distinguishable from unmethylated cytosine bases, and
introducing into the nucleic acid at least one base for generating a sequencing signal to be used as a reference signal for normalization, and
sequencing the nucleic acid, whereby a signal from each
   cytosine base of the nucleic acid, or
   guanine base of the nucleic acid
   and a reference signal from the at least on introduced base is obtained, and
normalizing the signal obtained from each
   cytosine base of the nucleic acid, or
   guanine base of the nucleic acid
   to the reference signal from the at least one introduced base.

In a preferred embodiment, the at least one introduced base is:
at least one base analog, and/or
at least one cytosine base, and/or
at least one guanine base.

In a preferred embodiment, the at least one introduced base is introduced into the nucleic acid as an at least one nucleotide.

In a preferred embodiment, the at least one nucleotide is introduced into the nucleic acid as an at least one oligonucleotide.

In a preferred embodiment, the at least one oligonucleotide comprises two to four, most preferably three bases for generating a sequencing signal.

In a preferred embodiment, the nucleic acid is treated with a bisulfite containing solution, whereby unmethylated cytosine bases of the nucleic acid are converted into sulfon-uracil bases or uracil bases whereas methylated cytosine bases remain unchanged.

In a preferred embodiment, the at least one base is introduced into the nucleic acid through an amplification reaction and/or a ligation reaction.

In a preferred embodiment, the bisulfite treated nucleic acid is amplified using at least one oligonucleotide that comprises at least one cytosine base and/or at least one base analog, whereby the at least one oligonucleotide is incorporated into a G-rich nucleic acid.

In a preferred embodiment, the at least one oligonucleotide further comprises a sequencing domain for hybridization of a sequencing primer, wherein the sequencing domain is located on the 5′ side of the at least one cytosine base and/or of the at least one base analog.

In a preferred embodiment, the bisulfite treated nucleic acid is amplified using at least one oligonucleotide that comprises at least one guanine base and/or at least one base analog, whereby the at least one oligonucleotide is incorporated into a C-rich nucleic acid.

In a preferred embodiment, the at least one oligonucleotide further comprises a sequencing domain for hybridization of a sequencing primer, wherein the sequencing domain is located on the 5′ side of the at least one guanine base and/or of the at least one base analog.

In a preferred embodiment, at least one oligonucleotide serves as a primer for the amplification reaction.

In a preferred embodiment, the at least one oligonucleotide comprises the at least one introduced base in a sequence part of the oligonucleotide that does not hybridize with the nucleic acid.

In a preferred embodiment, the sequence part of the oligonucleotide that does not hybridize with the nucleic acid is localized at the 5′ end of the oligonucleotide.

In a preferred embodiment, the at least one oligonucleotide comprises the at least one introduced base within a sequence part of the at least one oligonucleotide that hybridizes with the nucleic acid.

In a preferred embodiment, the sequence part of the at least one oligonucleotide is reverse complementary to the sequence of the nucleic acid.

In a preferred embodiment, the sequence part of the at least one oligonucleotide that hybridizes with a sequence of the nucleic acid has a length of between 15 to 30 nucleotides.

In a preferred embodiment, the amplification reaction is mediated by a polymerase, preferably by a heat stable polymerase.

In a preferred embodiment, the at least one oligonucleotide is introduced into the nucleic acid through ligation, with the at least one oligonucleotide-comprising:
at least one cytosine base and/or at least one base analog for sequencing a G-rich strand of the nucleic acid, or
at least one guanine base and/or at least one base analog for sequencing a C-rich strand of the nucleic acid.

In a preferred embodiment, the at least one oligonucleotide comprises a sequencing domain for hybridization of a sequencing primer that is located on the 5′ side of the at least one cytosine base and/or at least one base analog or guanine base and/or at least one base analog.

In a preferred embodiment, the sequencing reaction is performed using one kind of labeled dideoxyribonucleoside triphosphates that forms base pairs with either
the cytosine bases of the nucleic acid and the introduced base, or
the guanine bases of the nucleic acid and the introduced base.

In a preferred embodiment, the at least one signal stemming from the at least one introduced base is identified.

In a preferred embodiment, the area under the sequencing curve is determined for each signal stemming from a cytosine base or a guanine base of the nucleic acid and for each signal stemming from the at least one introduced base, to yield area values.

In a preferred embodiment, normalization occurs either by dividing the area value of each signal stemming from a cytosine base or a guanine base of the nucleic acid by the area value of the signal from the at least one introduced base, or by dividing the height value of each signal stemming from a cytosine base or a guanine base of the nucleic acid by the height value of the signal from the at least one introduced base.

In a preferred embodiment, the percentage of methylation of a specific position is obtained by calibrating the normalized signal of the nucleic acid to be analyzed against the normalized signal of a reference nucleic acid.

In a preferred embodiment, the nucleic acid is genomic DNA.

Subject matter of the invention is also a method for determining the clonality of a sample, comprising

a) isolating genomic DNA from a sample;
b) submitting the isolate genomic DNA to the method of claim 1, wherein normalized signals for cytosine bases or guanin bases are obtained,
c) deducing that a sample is of monoclonal origin wherein only cytosine bases or guanine bases are detected that are specific for either the maternal chromosome or the paternal chromosome; or deducing a sample is of polyclonal origin, wherein cytosine bases or guanine bases are detected that are specific for the maternal as well the paternal chromosome.

An additional subject matter of the invention is also an oligonucleotide for amplifying a nucleic acid, comprising
a first sequence part that is reverse complementary to the nucleic acid to be amplified for initiating an amplification reaction,
a second sequence part that contains at least one base for generating a sequencing signal to be used for the normalization of sequencing signals stemming from the nucleic acid, and
a third sequence part for the hybridization of a sequencing primer. Preferably the second and third sequence part are not identical. In particular said at least one oligonucleotide is suitable for amplification.

A further subject matter of the invention is also a kit for the realization of the method of the invention, with the following components:
a) a chemical reagent or an enzyme which alters the base pairing behavior of methylated cytosine bases and/or unmethylated cytosine bases of the nucleic acid such that methylated cytosine bases become distinguishable from unmethylated cytosine bases,
b) at least one oligonucleotide that comprises at least one cytosine base and/or at least one guanine base and/or at least one base analog, and
c) an enzymatic activity for amplifying a nucleic acid using the at least one oligonucleotide as a primer and/or an enzymatic activity for ligating the at least one oligonucleotide to a nucleic acid.

A preferred kit is a kit, wherein the chemical reagent is bisulfite.

A preferred kit is a kit, wherein at least one oligonucleotide is an oligonucleotide comprising
a first sequence part that is reverse complementary to the nucleic acid to be amplified for initiating an amplification reaction,
a second sequence part that contains at least one base for generating a sequencing signal to be used for the normalization of sequencing signals stemming from the nucleic acid, and
a third sequence part for the hybridization of a sequencing primer. Preferably the second and third sequence part are not identical. In particular said at least one oligonucleotide is suitable for amplification.

Subject matter of the invention is also the use of the method of the invention or of an oligonucleotide according to the invention or of a kit according to the invention for diagnosis and/or prognosis of adverse events for patients or individuals, whereby these adverse events belong to at least one of the following categories:
undesired drug interactions; cancer diseases; CNS malfunctions; damage or disease; symptoms of aggression or behavioral disturbances; clinical; psychological and social consequences of brain damages; psychotic disturbances and personality disorders; dementia and/or associated syndromes; cardiovascular disease, malfunction or damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, of the muscles, of the connective tissue or of the bones; endocrine and metabolic malfunction, damage or disease; headaches or sexual malfunction.

According to the invention it is also preferred to use the method of the invention or an oligonucleotide according to the invention or a kit according to the invention, for distinguishing cell types and/or tissues and/or for investigating cell differentiation.

According to the invention it is also preferred to use the method of the invention or an oligonucleotide according to the invention or a kit according to the invention, for identifying an indication-specific target, wherein an indication-specific target in a nucleic acid is defined by a difference in the methylation rate of a nucleic acid derived from a diseased tissue in comparison to the methylation rate of a nucleic acid derived from a healthy tissue.

### DESCRIPTION OF THE DRAWINGS

FIG. 1:

FIG. 1 describes the complete conversion of unmethylated cytosine to uracil, also referred to as bisulfite conversion, which is known in the art. In the first step of this reaction, unmethylated cytosine bases are sulfonated at position C6 at a pH around 5 through reaction with hydrogensulfite.

The second step is the deamination that takes place rather spontaneously in aqueous solution. Thereby, cytosine sulfonate is converted into uracil sulfonate. The third step is the desulfonation step, which takes place in alkaline conditions, resulting in uracil.

FIGS. 2 and 3:

FIG. 2 shows data processing of the sequenced TFF1 gene PCR product. The sequencing electorpherogram of 50% methylated, bisulfite treated DNA is depicted.

FIG. 2 two contains three horizontal panels, denoted A, B, and C, all showing electro-pherograms. In all panels A, B, and C, the run time is represented on the x-axis and the signal intensity on the y-axis. CpG sites are denoted by arrows.

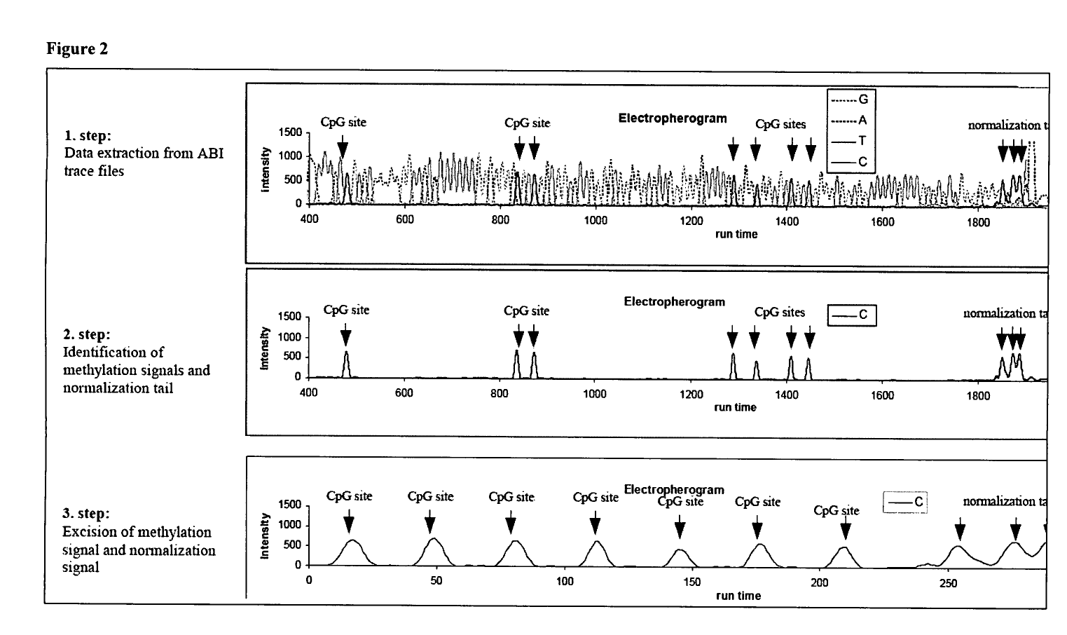

The upper panel A of FIG. 2 shows the data that was exported from ABI trace files after completed sequencing of a nucleic acid that is to be analyzed for its methylation rate. Each of the four lanes represents the signal for one base (A, C, G, T).

Panel B shows the identified methylation signals stemming from the nucleic acid and the signals stemming from the at least one introduced base of an at least one oligonucleotide. In the example shown, the at least one oligonucleotide that was used was introduced into the bisulfite-treated nucleic acid as a primer in a PCR amplification and contained three cytosine bases. The three cytosine bases were introduced into the nucleic acid to be analyzed in a PCR reaction. All cytosine bases of the nucleic acid as well as the three introduced cytosine bases are detected using the same sequencing dye.

Panel C shows the excised methylation and normalization signals. As can be seen, the use of only one dye for cytosine is sufficient to perform determination of the methylation rate of a nucleic acid.

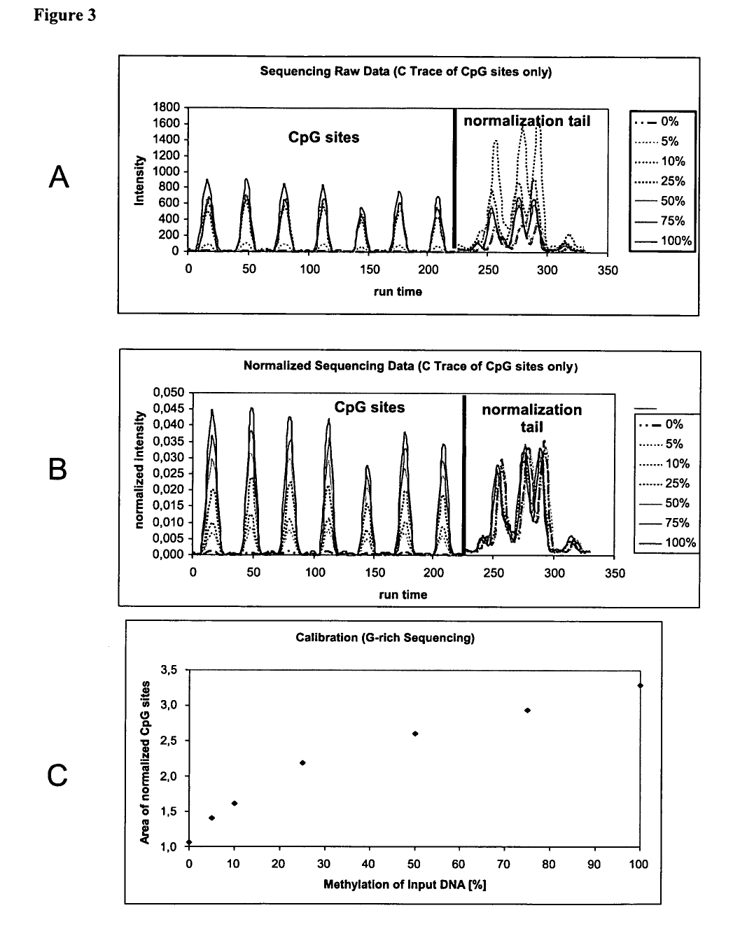

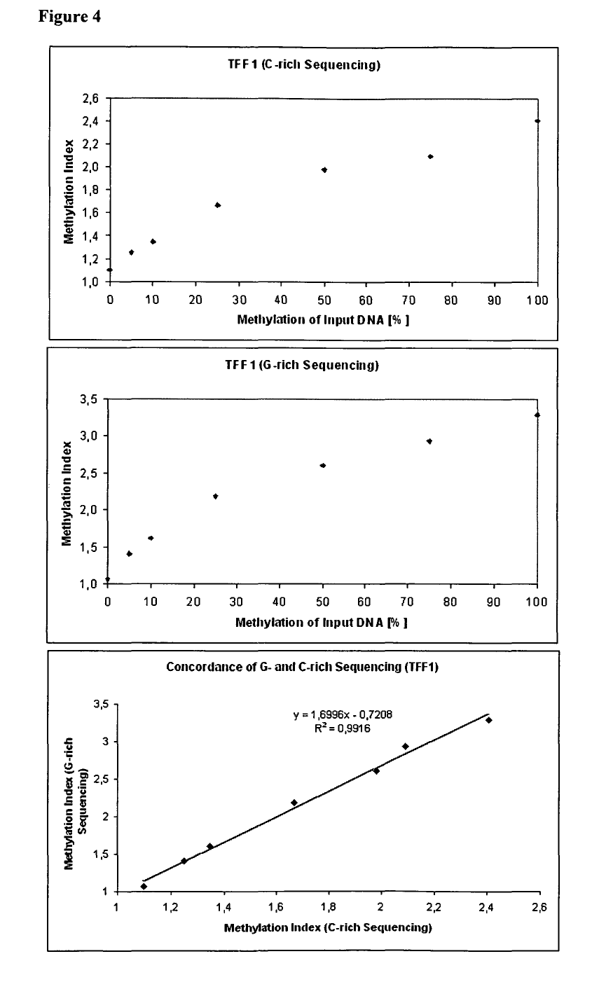

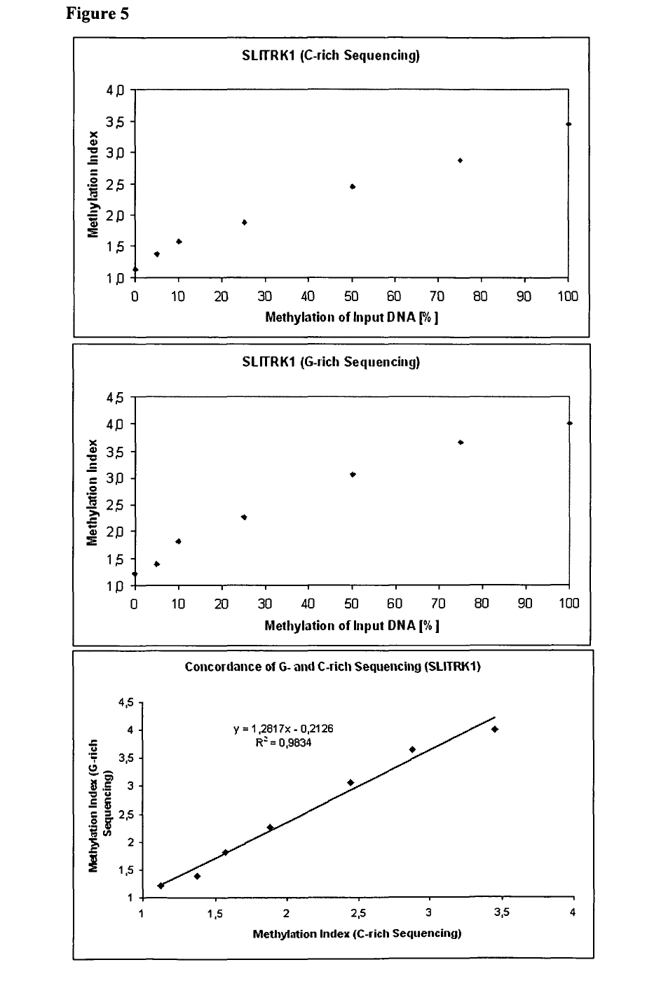

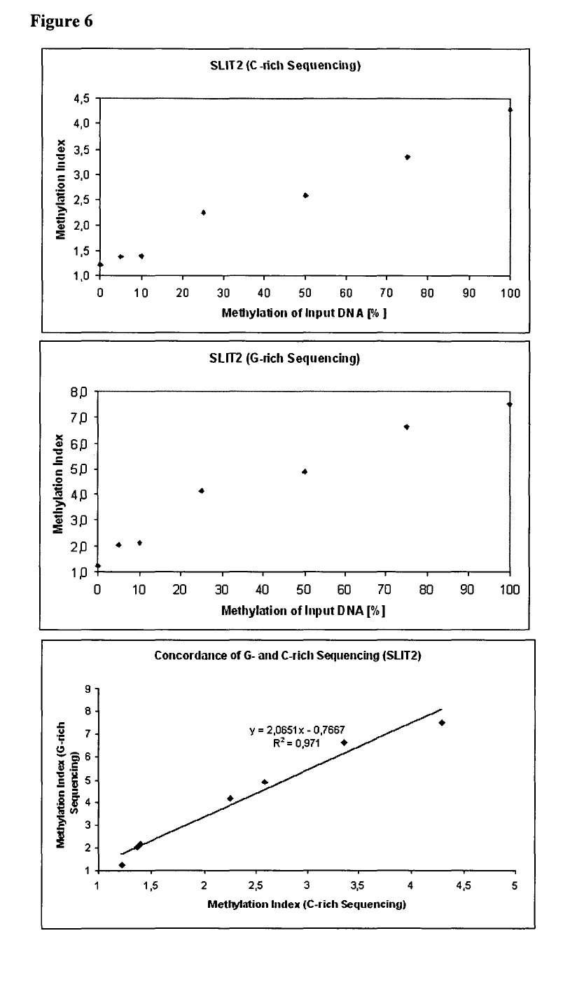

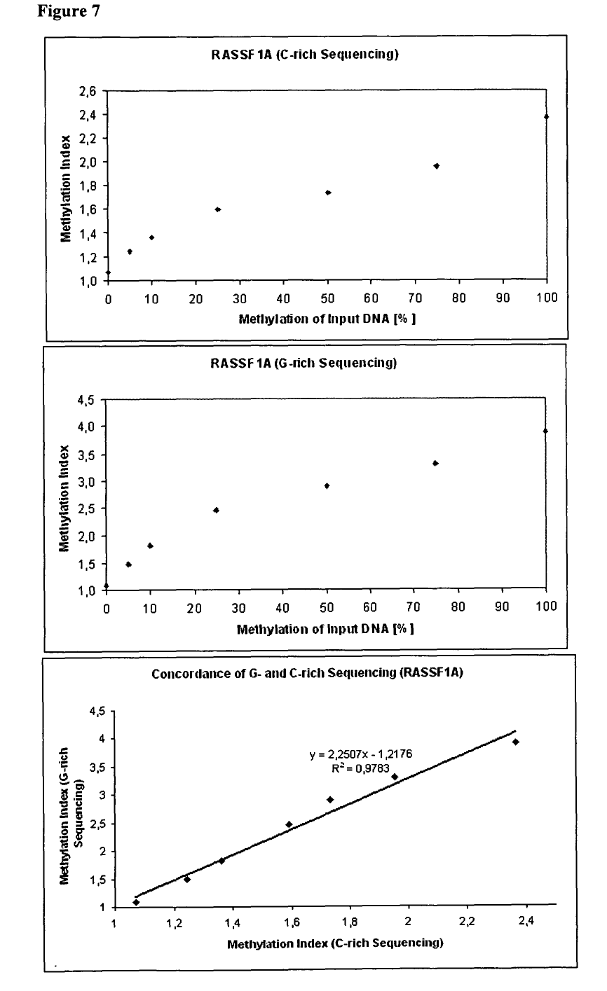

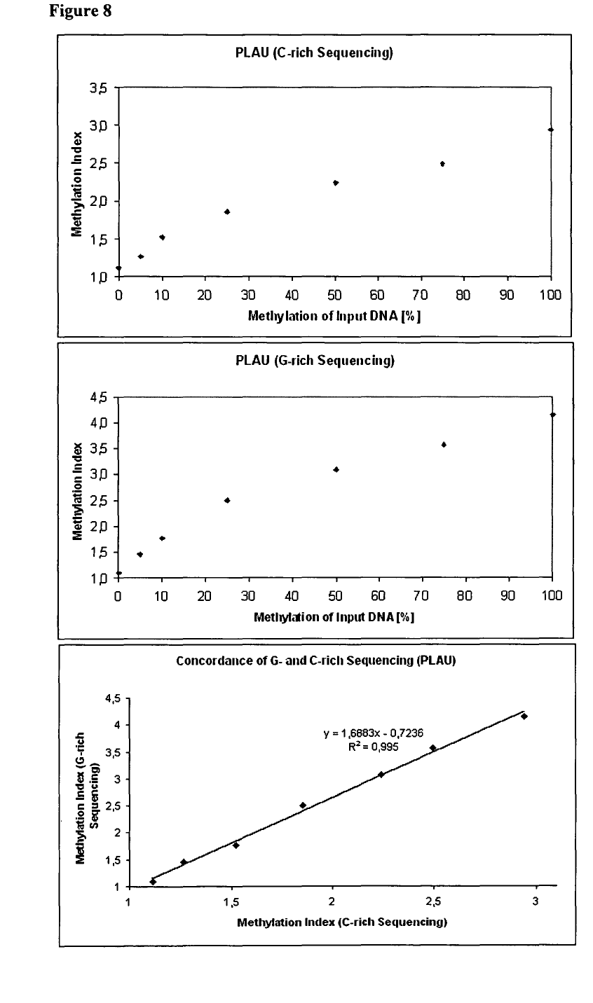

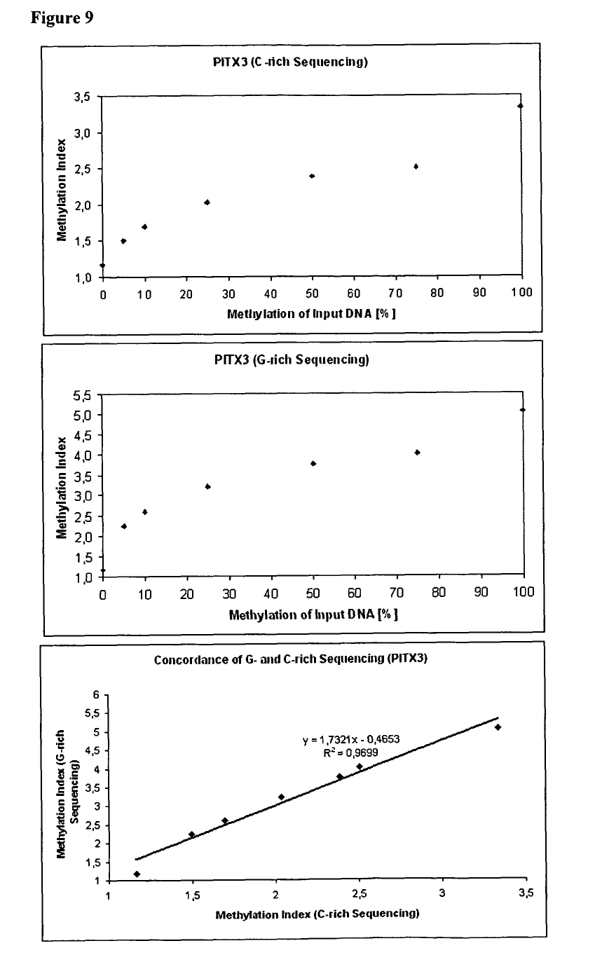

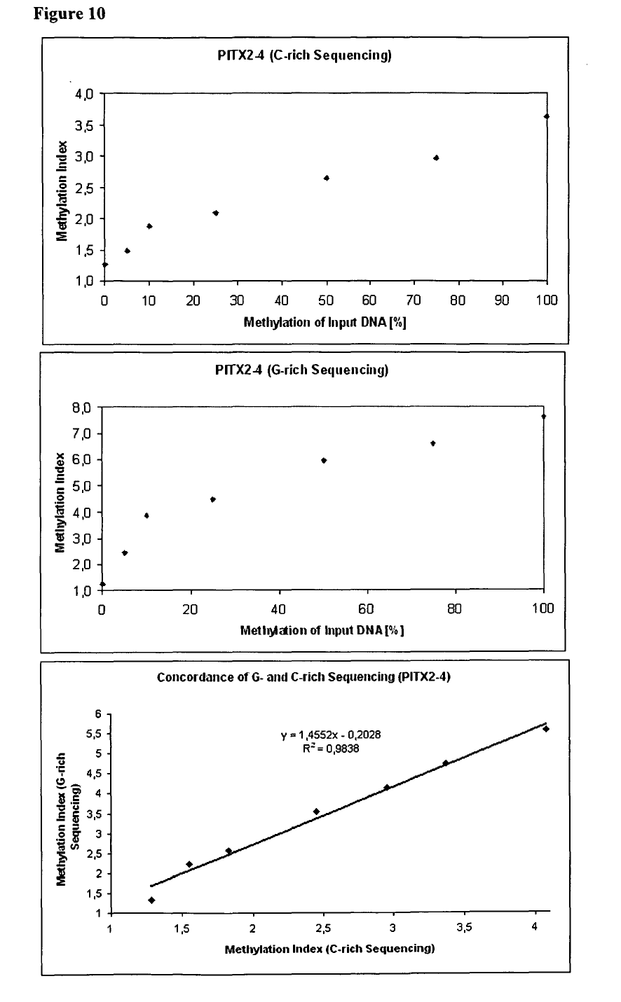

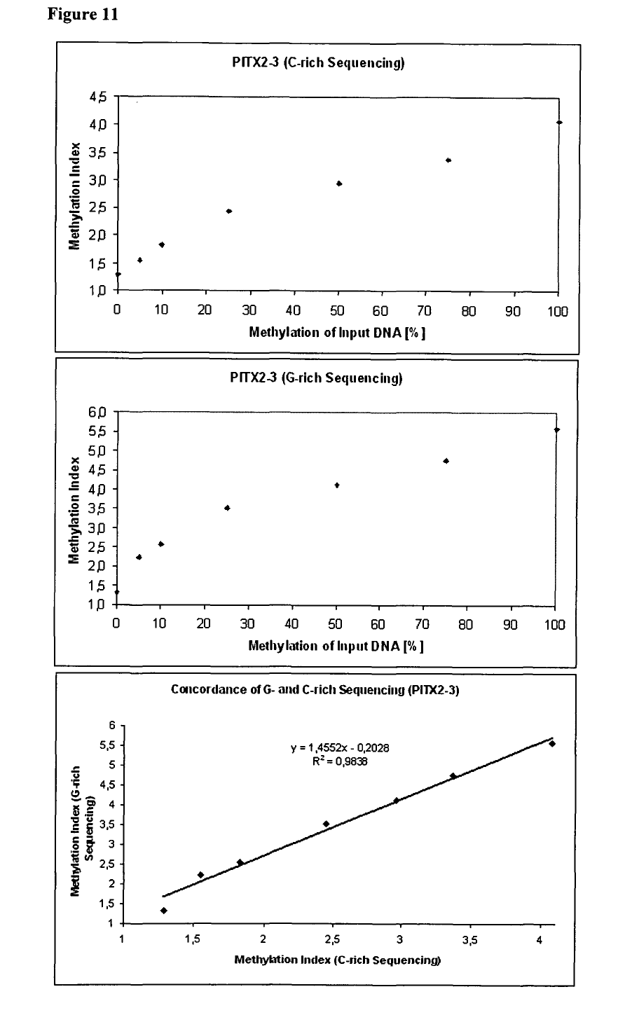

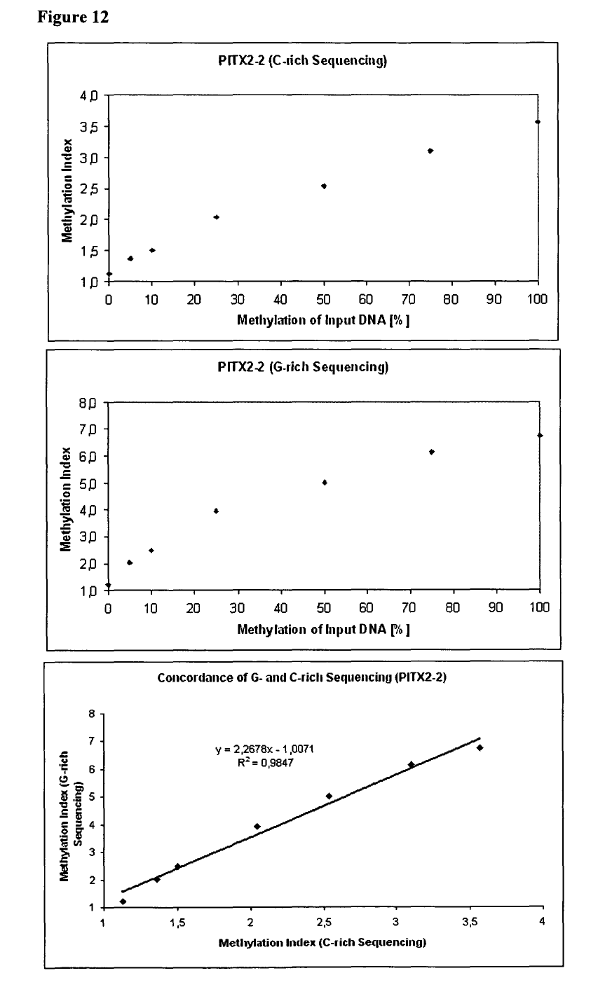

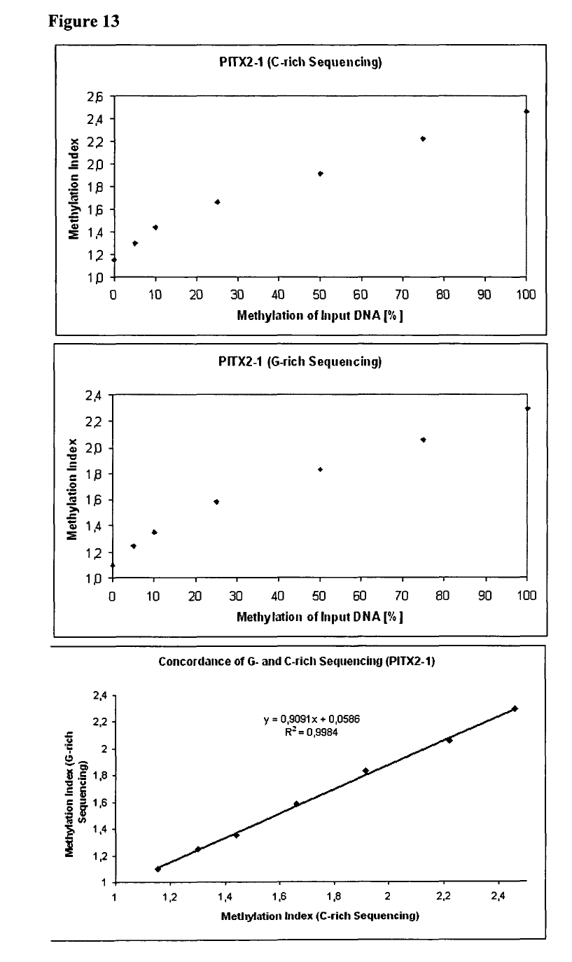

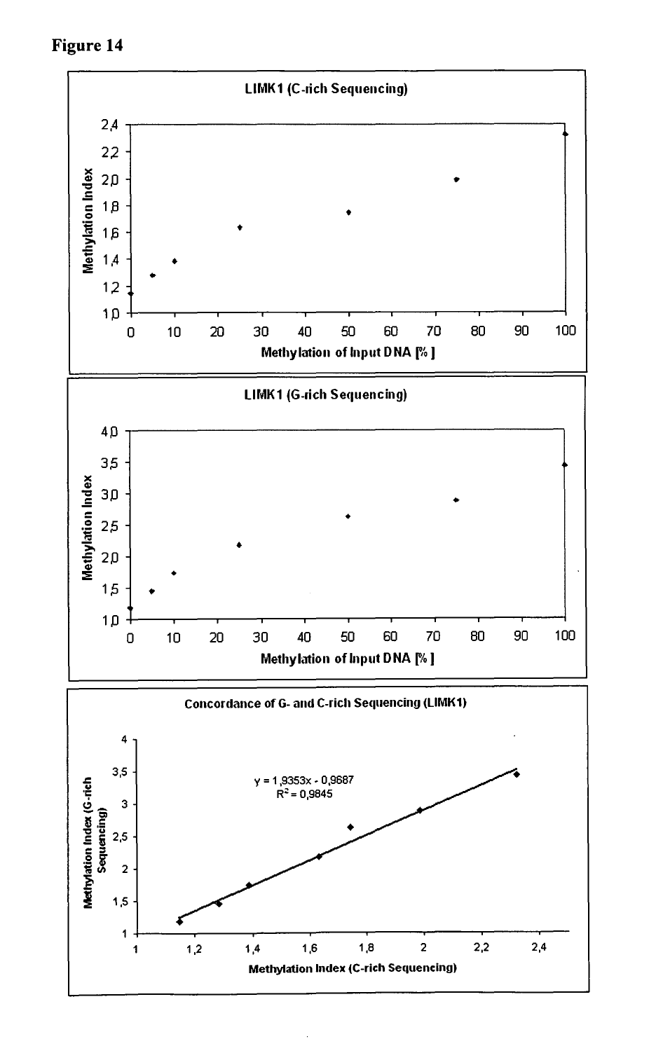

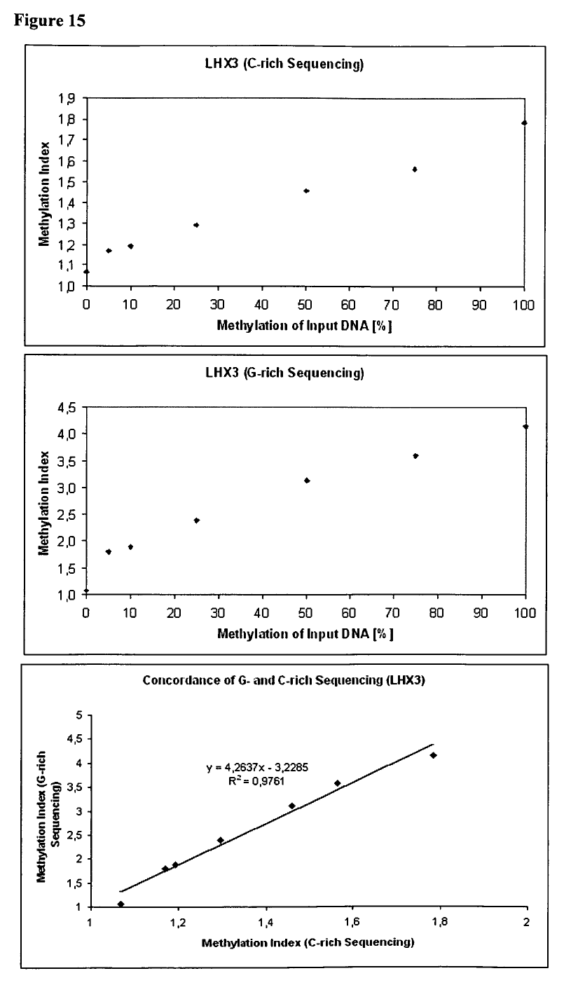

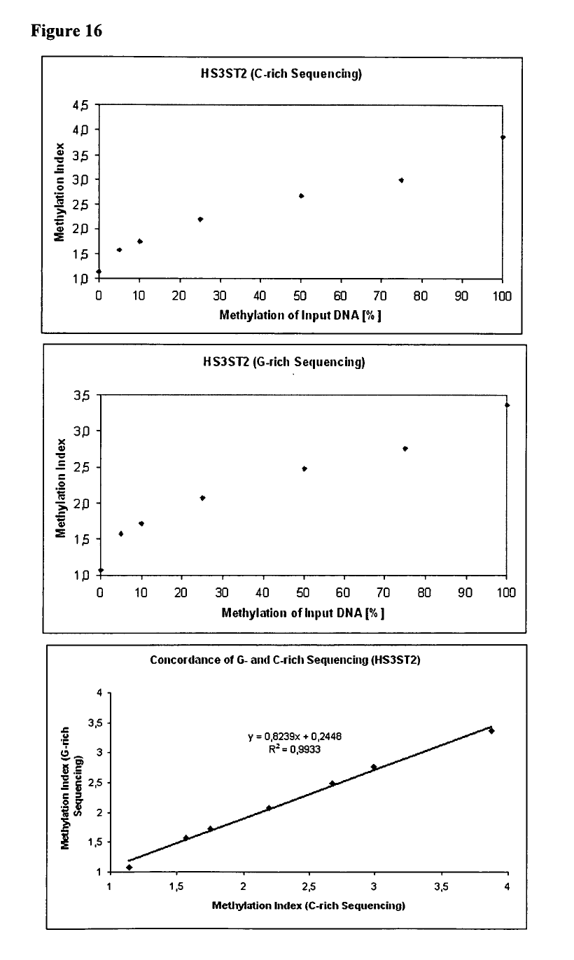

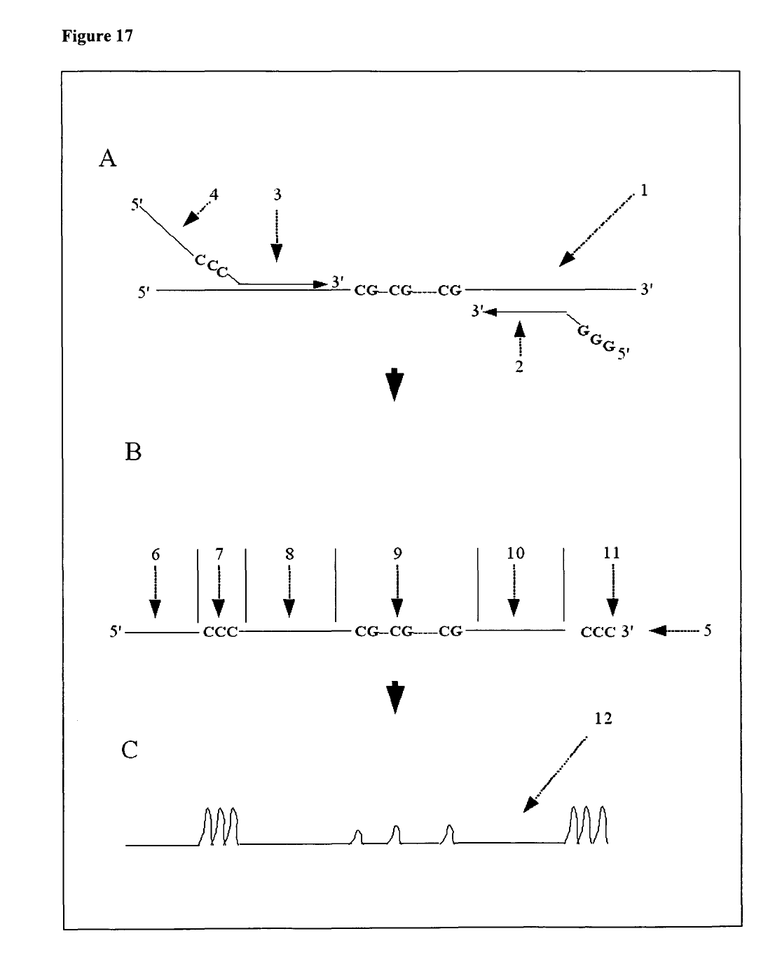

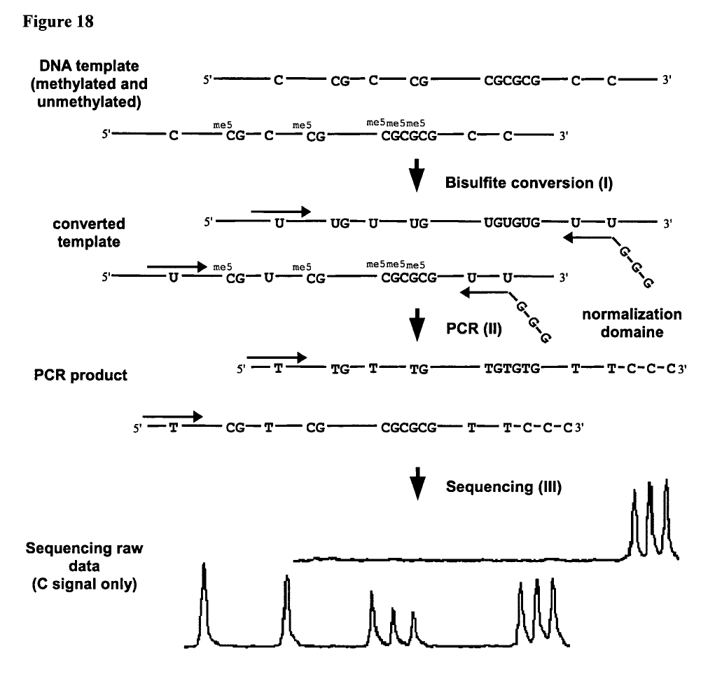

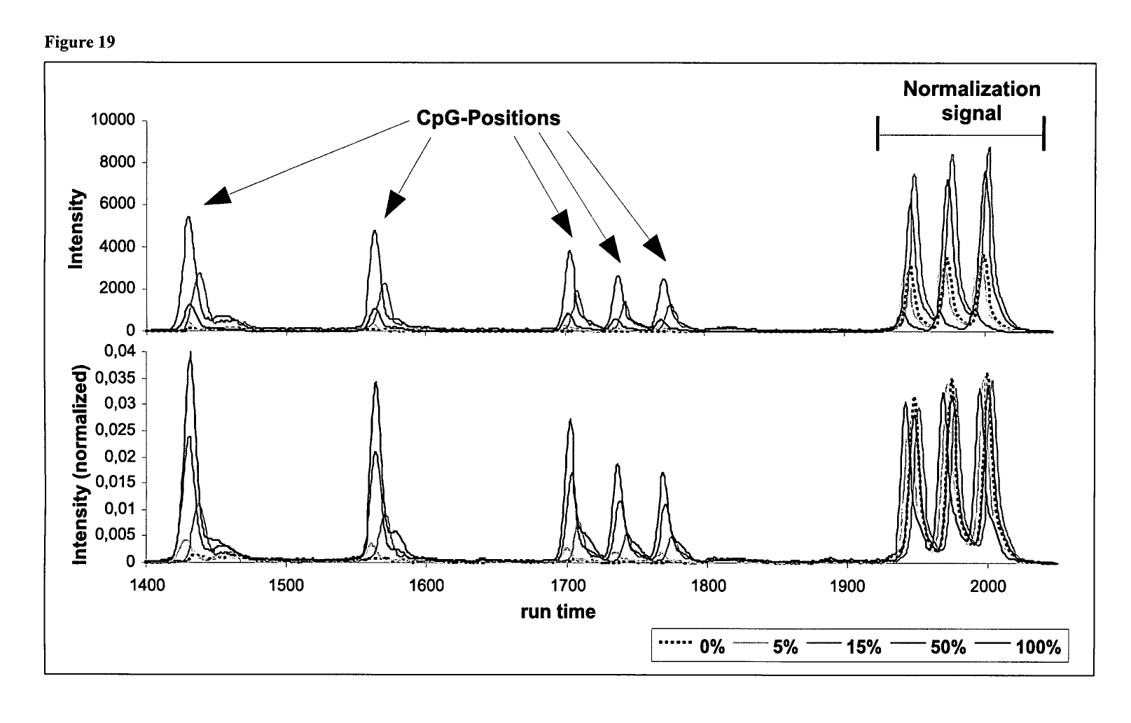

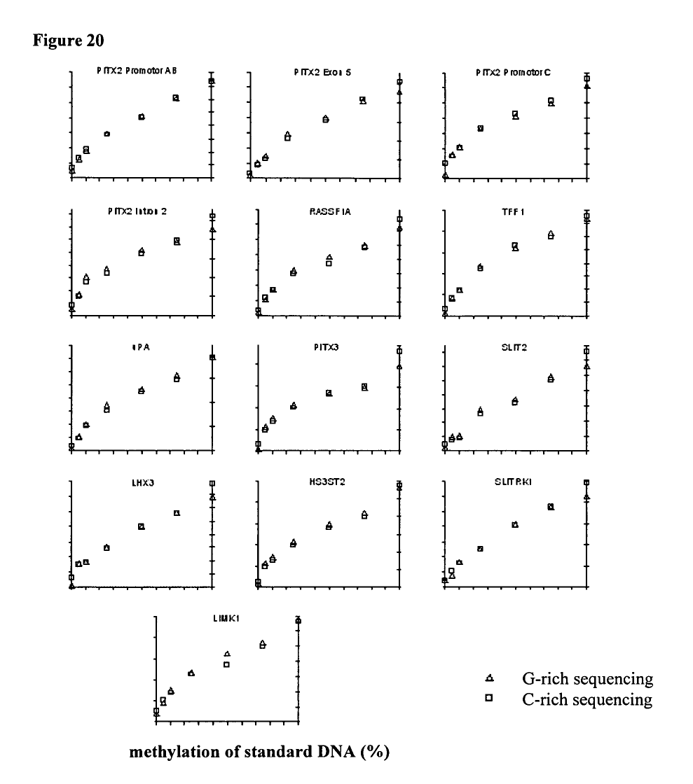

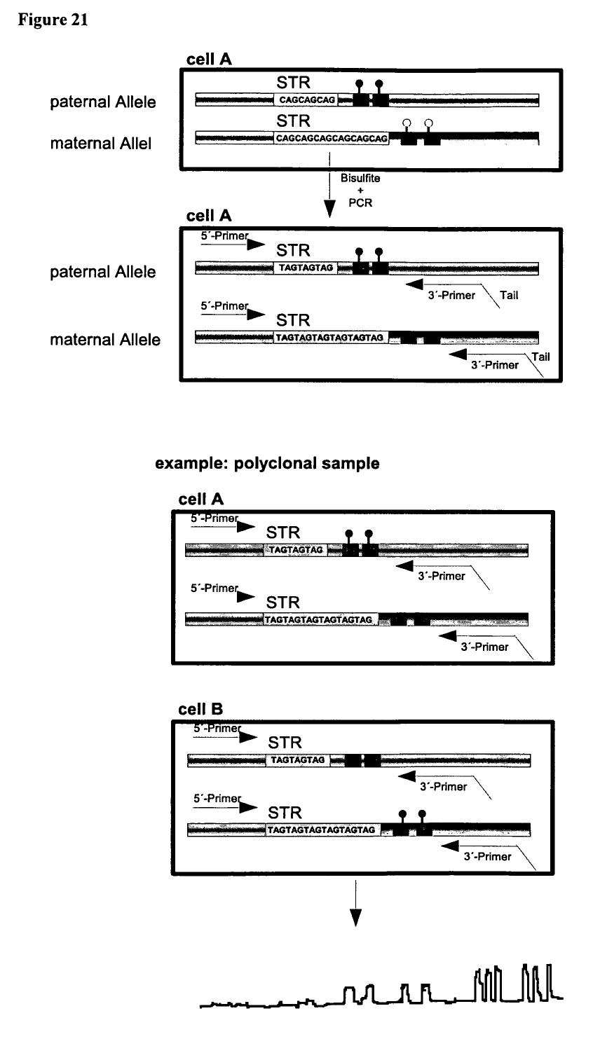

The process of data analysis of the TFF1 gene (forward [G-rich] primer) is exemplarily depicted in FIG. 2 (together with FIG. 3). Sequence data from the 50% methylated DNA was exported from the “.ABI” trace files generated by the software of the sequencing machine and the trace of the C signal (containing the methylation signal and the normalization tail) is depicted (FIG. 2, panel A and B). The signals at the CpG sites and the normalization tail were identified and excised (FIG. 3, B and C).

FIG. 3 shows data normalization. Panel A (top) shows the sequencing raw data (cytosine trace of CpG sites only; Intensity over run time), panel B shows the normalized sequencing data (cytosine trace of CpG sites only; normalized intensity over run time), panel C shows the calibration (G-rich sequencing only; area of normalized CpG sites over methylation of input DNA in %).

In panels A and B, different ratios of methylated to unmethylated nucleic acid were analyzed, namely 0%, 5%, 10%, 25%, 50%, 75%, 100% methylated DNA, shown in different shades.

Peak intensities at CpG sites were divided by the area of the normalization tail ([A] and [B]). The integrated areas of all normalized signals (methylation index) reflect the average methylation rate of the analyzed sequence [C].

FIG. 3 (A) shows the cytosine traces of all used standard DNAs (0%, 5%, 10%, 25%, 50%, 75% and 100% methylated). CpG sites and the normalization tail were already excised in FIG. 3. Normalization was performed by dividing the area of all CpG sites by the area of the normalization tail (FIG. 3, B). Quantitative methylation information is reflected by the normalized area (methylation index) of the CpG sites (FIG. 3, C).

FIGS. 4 to 16:

FIGS. 4 to 16 show the results of the sequencing of the 0%, 5%, 10%, 25%, 50%, 75% and 100% methylated DNA mixtures using 13 PCR amplicons (10 different genes). All amplicons were sequenced using forward and reverse primers (Table 1). The methylation rate (methylation index) is displayed by the normalized areas of the peaks of the respective methylation sites that were normalized against the normalization tail. The methylation indices of all 13 amplicons and both sequencing directions for each amplicon show a strong correlation to the level of methylation of the input DNA.

All FIGS. 4 to 16 consist of three panels: panel A (top), panel B (middle), and panel C (bottom). Panel A always shows C-rich sequencing (bisulfite anti-sense strand) of a particular gene (methylation index over methylation of input DNA in %). Panel B always shows G-rich sequencing (bisulfite sense strand) of a particular gene (methylation index over methylation of input DNA in %). Panel C always shows the concordance of G-rich and C-rich sequencing of a particular gene (methylation index of the G-rich sequencing over methylation index of the C-rich sequencing).

FIG. 4:

FIG. 4 shows the results of the sequencing of the TFF1 gene.

FIG. 5:

FIG. 5 shows the results of the sequencing of the SLITRK1 gene.

FIG. 6:

FIG. 6 shows the results of the sequencing of the SLIT2 gene.

FIG. 7:

FIG. 7 shows the results of the sequencing of the RASSF1A gene.

FIG. 8:

FIG. 8 shows the results of the sequencing of the PLAU gene.

FIG. 9:

FIG. 9 shows the results of the sequencing of the PITX3 gene.

FIG. 10:

FIG. 10 shows the results of the sequencing of the PITX2 (region 4) gene.

FIG. 11:

FIG. 11 shows the results of the sequencing of the PITX2 (region 3) gene.

FIG. 12:

FIG. 12 shows the results of the sequencing of the PITX2 (region 2) gene.

FIG. 13:

FIG. 13 shows the results of the sequencing of the PITX2 (region 1) gene.

FIG. 14:

FIG. 14 shows the results of the sequencing of the LIMK1 gene.

FIG. 15:

FIG. 15 shows the results of the sequencing of the LHX3 gene.

FIG. 16:

FIG. 16 shows the results of the sequencing of the HS3ST2 gene.

FIG. 17:

FIG. 17 shows a preferred embodiment of the method for determining the methylation rate of a nucleic acid through sequencing, in which two oligonucleotides are used as primers to introduce bases into a nucleic acid to be analyzed via a PCR amplification reaction.

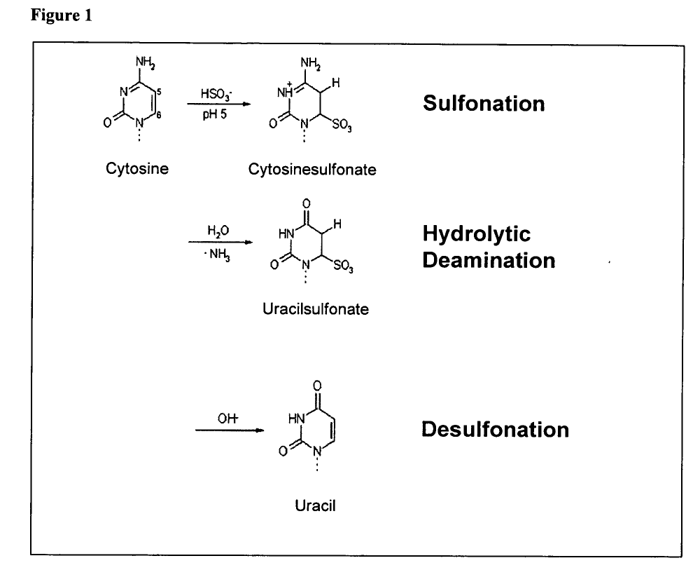

In panel A, a nucleic acid 1 is shown that was treated such that unmethylated cytosines were converted into uracils. This was done using bisulfite treatment, such as described with reference to FIG. 1.

Of the bisulfite-treated nucleic acid 1, the bisulfite sense strand is depicted. Due to bisulfite treatment, this DNA strand 1 is rich in guanine bases compared to cytosine bases (G-rich strand). In the middle of the nucleic acid molecule 1, three CpG sites are located 9, which will be analyzed using the method according to this invention.

The nucleic acid strand 1 is amplified using an enzymatic amplification reaction in the form of a polymerase chain reaction (PCR). Two oligonucleotides 2, 3 are used in the PCR.

Both of the oligonucleotides (primers) 2, 3 shown here for amplifying a nucleic acid 1 comprise a first sequence part that is reverse complementary to the nucleic acid to be amplified which serves for initiating an amplification reaction, a second sequence part that contains at least one base (C or G in this examples) for generating a sequencing signal to be used for the normalization of sequencing signals stemming from the nucleic acid, and a third sequence part for the hybridization of a sequencing primer. Due to the fact that the second and third sequence parts are not identical, the at least one base for generating a sequencing signal will be sequenced before the nucleotides of interest of the nucleic acid to be analyzed, which simplifies the determination of the methylation rate.

A first oligonucleotide 2 is a C-rich primer that comprises a sequence part at its 3′ end that hybridizes to the nucleic acid strand 1 and takes part in initiating the amplification reaction. The 5′ end of the first primer 2 comprises at least one guanine base. Therefore, the first primer 2 amplifies the bisulfite anti-sense strand, introducing at least one guanine base as a reference base for sequencing.

A second oligonucleotide 3 is a G-rich primer that comprises a sequence part that hybridizes to the nucleic acid strand 1, which is located at the 3′ side of the second primer 3. On the 5′ side to the sequence part that hybridizes to the nucleic acid strand 1, at least one cytosine base is located. On the 5′ end of the second primer 3, a sequencing domain 4 for hybridization of a sequencing primer is located. The second primer 3 amplifies the bisulfite sense strand, introducing at least one cytosine base as reference base for sequencing.

A sequencing primer used for sequencing the nucleic acid 1 can hybridize to the sequencing domain 4 of the second primer 3 to initiate a sequencing reaction, such as a cycle sequencing reaction. In the example shown in FIG. 17, the first primer 2 contains three guanines and the second primer 3 contains three cytosine bases that generate a reference signal when sequenced. These reference signals will later be used for normalizing the signals stemming from guanine of cytosines of the nucleic acid 1.

Due to the fact that both the first (reverse) primer 2 and the second (forward) primer 3 comprise at least one guanine or cytosine base as an introduced base, respectively, the PCR product contains at least one guanine or cytosine base at each side, i.e. at the 5′ and the 3′ end. This is advantageous for x-axis normalization.

Panel B shows an amplification product 5 of a PCR amplification reaction of a nucleic acid 1 using first and second primers 2, 3 as described with reference to FIG. 17 A. The strand shown of the amplification product 5 is the bisulfite sense strand (G-rich strand); the bisulfite anti-sense strand (C-rich strand) is not shown.

The depicted amplification product 5 comprises, in 5′ to 3′ direction: a sequencing domain 6, cytosine reference bases 7 to be used for normalization, the sequence that the second primer 3 hybridized with 8, the analyzed sequence of the nucleic acid 9 containing the methylation information, the sequence 10 that the first primer 2 hybridized with, and cytosine reference bases 11 to be used for normalization.

It is noted that the three guanine bases of the first primer 2 are now introduced into the amplification product 5 of the bisulfite sense strand as cytosines. Thus, the amplification product 5 contains reference signals for normalization at both its 5′ and 3′ end, which is advantageous for determining the methylation rate of the nucleic acid 1.

Panel C depicts the sequence trace data 12 from the cytosine signal, which was generated form the sequencing data of the PCR amplification product 5 shown in panel B of FIG. 17. Signals stemming from cytosines from the oligonucleotides in the form of primers are located on the 5′ and the 3′ side of the CpG sites of interest, making the determination of the methylation rate of the cytosine bases to be analyzed 9 easy, reliable and reproducible.

FIG. 18 illustrates the principle of the method of the invention on the example of a G-rich sequencing via the forward primer. (I) Bisulfite conversion: unmethylated cytosines deaminates to uracil while methylated cytosines remain unchanged. (II) PCR amplification by means of reverse primer with normalization domain at its 5′ end, said domain has three guanosine base which signal serve for normalization (normalization signal). Therefore, three cytosine residues are incorporated into the ends of the PCR products. (III) Sequencing. The sequencing histograms of the each PCR product now comprise a normalization signal independent form the methylation of the original DNA. Thereby, only the cytosine sequencing signal is considered.

FIG. 19 shows the principle of normalization according to the method of the invention. Shown are the sequence histograms (only cytosine signals) of 5 different methylation mixtures—in the upper part before normalization and in the lower part after normalization. The normalization signal is identified for each sample. The intensities of each measured value are then divided by the area of the normalization signal. The heights as well as the areas of the peaks at the CpG positions correlate with the methylation of the methylation mixtures after normalization.

FIG. 20: Dependency of the normalized methylation signals (sum of all normalized peak areas) on the methylation of the applied standard DNA. 13 different loci were sequenced by means of both a guanosine-rich forward primer (primary y-axis) and a cytosine-rich reverse primer (secondary y-axis). This figure is equivalent to the content of FIGS. 4-16 summarizing the data.

FIG. 21: Comparison of the method of the invention with correspondent QM assays. Shown are the results obtained by the analysis of the three genes PITX2, uPA and TFF1. The analyzed standard DNA samples were characterized by a methylation of 0, 5, 10, 25, 50, 75 and 100%. The displayed measured values are median values of three different QM assay runs±standard deviation (secondary y-axis) and the results of a single sequencing run by means of guanine-rich primer according to the method of the invention (primary y-axis), respectively.

FIG. 22 illustrates a preferred embodiment of the invention, wherein the clonality of a sample, in particular of tumor cells is determined. The human chromosome set is a duplicate set, wherein one copy of a gene is derived from the mother (maternal allele) and the other from the father (paternal allele). One allele is only active in each cell, while the other allele is inactivated by methylation. Maternally and paternally derived chromosomal regions can be differentiated for example by the different lengths of short tandem repeats. In case of a monoclonal sample, the sample will only generate a sequencing histogram according to the invention that reflects the methylated cytosines of either the maternal or the paternal allele. In case of a polyclonal sample, the sample will generate a sequencing histogram according to the invention that reflects the methylated cytosines of the maternal as well the paternal allele. When the sample comprises tumor cells, it is possible to determine the clonality of the cancerous cells. Thereby it is determined if the cancerous cells are derived from a single origin or from multiple origins.

### EXAMPLES

### Example 1

In the first step according to the method of this invention, the nucleic acid to be analyzed is incubated with a chemical reagent or an enzyme containing solution, whereby unmethylated cytosine bases are converted into uracil bases. Accordingly, bisulfite treated DNA comprises only a small number of cytosine bases and all remaining cytosine bases derive from previously methylated cytosine bases.

In the second step, at least one base is introduce into the bisulfite-treated nucleic acid. This is done in this example using enzymatic amplification reaction, which is performed with at least one oligonucleotide, which comprises at least one cytosine base for amplifying the bisulfite sense strand or at least one guanine base for amplifying the bisulfite anti-sense strand of the nucleic acid. The mixture is then incubated, whereby the nucleic acid is amplified. In the given examples, amplification is performed using the polymerase chain reaction (PCR).

In order to quantify the rate of DNA methylation, bisulfite-treated DNA is amplified in this example using at least one oligonucleotide in the form of a tagged primer, which leads to an incorporation of additional cytosine bases at the end of the PCR product of the bisulfite sense strand. If this strand is sequenced (G-rich sequencing) in a later step, these additional cytosine bases can be used as reference signals to quantify the methylation rate of the DNA.

In the given examples, the at least one oligonucleotide comprises three cytosine bases for amplifying the bisulfite sense strand or three guanine bases for amplifying the bisulfite anti-sense strand.

In the oligonucleotides used in the examples given here, the at least one cytosine or guanine base is located at the 5′ end of the oligonucleotide, and on the 3′ side to that 5′ end of the oligonucleotide, a nucleotide sequence is located which specifically hybridizes with the particular target gene of the nucleic acid that is to be analyzed. Based on the individual sequence part at the 5′ tail end that comprises at least one cytosine or guanine base, the oligonucleotides used in these examples will generate a sequencing reference signal that can be used to normalize the sequencing signals obtained for the cytosine or guanine base of the nucleic acid that is analyzed.

The 5′ end of the oligonucleotides that serve as primers in the PCR bear either the sequence ACTCC (when used for amplifying the bisulfite sense strand) or AGGTG (when used for amplifying the bisulfite anti-sense strand) (see Table 1).

In contrast to the bisulfite sense strand, the bisulfite anti-sense strand, which is generated during PCR amplification, contains few guanine bases and all remaining guanine bases reflect previously methylated CpG sites. Accordingly, guanine bases have to be incorporated (here using tagged primers) at one terminal end of the PCR amplification product in order for determining the methylation rate of this strand using sequencing (C-rich sequencing).

### Materials and Methods

### DNA Preparation

Unmethylated DNA was prepared by MDA (multiple displacement amplification), a genome-wide amplification method described by Dean F B, Hosono S, Fang L, Wu X, Faruqi A F, Bray-Ward P, Sun Z, Zong Q, Du Y, Du J, Driscoll M, Song W, Kingsmore S F, Egholm M and Lasken R S (2002) Comprehensive human genome amplification using multiple displacement amplification. Proc. Natl. Acad. Sci. USA, 99, 5261-5266).

Methylated DNA was prepared by treating unmethylated DNA (obtained as described above) with SssI methyltransferase (New England Biolabs) in the presence of S-adenosylmethionine according to the manufacturer's instructions.

DNA quantification took place using the UV spectrophotometer NanoDrop ND-1000 (NanoDrop Technologies, DE USA).

Mixtures of both methylated and unmethylated DNA were prepared reflecting 0%, 5%, 10%, 25%, 50%, 75% and 100% total methylation. Bisulfite treatment was performed as previously described (Berlin K, Ballhause M, Cardon K. Improved bisulfite conversion of DNA. 2005; PCT/WO/EP/05/038051)

### PCR

13 regions within 10 different genes were PCR amplified using two oligonucleotides in the form of primers specific for bisulfite-converted DNA (Table 1). All primers contained a tag at their 5′-end in order to introduce the normalization signal into the PCR products. Due to the character of bisulfite-converted DNA, the gene specific sequences of the forward primers contained no cytosine bases, whereas the gene specific sequences of the reverse primers contain no guanine bases. Therefore, the normalization tag for the forward primers contained cytosine or guanine bases for the reverse primers, respectively.

It is noted that it is also possible to introduce the at least one cytosine base or guanine bases as a mismatch within the sequence of the at least one oligonucleotide, rather than as a tail as described in these examples.

PCR amplification reactions were performed in a total volume of 25 μl containing 5 ng template DNA, 1 U Hotstar Taq polymerase (Qiagen), 12.5 μmol of forward and reverse primer, 1×PCR buffer (Qiagen), 0.2 mmol/l of each dNTP (Fermentas). Cycling was performed using a Mastercycler (Eppendorf) under the following conditions: 15 min at 95° C. and 45 cycles at 95° C. for 20 s, 58° C. for 45 s and 72° C. for 30 s.

### Bisulfite Sequencing

Digestion of remaining dNTPs and primers was performed in a total volume of 7 μl, containing 5 μl PCR product and 2 μl ExoSAP-IT (Amersham Bioscience) at 37° C. for 45 min. The enzyme was inactivated by heating to 95° C. for 15 min. ExoSAP-IT digested PCR products were subjected to the cycle sequencing reaction. Cycle sequencing reaction was carried out in a total volume of 20 μl containing 5 μl ExoSAP-IT™ digested PCR product, 1× sequencing buffer, 1 μl BigDye Terminator v3.1™ (Applied Biosystems) and 0.5 μmol/l primer. Cycling was performed using a Mastercycler (Eppendorf) under the following conditions: 2 min at 96° C. and 25 cycles at 96° C. for 30 s, 55° C. for 15 s and 60° C. for 4 min. All PCR products were sequenced using the G-rich forward primer (G-rich sequencing) and the C-rich reverse primer (C-rich sequencing).

Cycle sequencing products were purified using DyeEx™ 96 plates (Qiagen) according to the manufacturer's instructions. Sequence analysis of the purified cycle sequencing product was performed with the 3730 DNA Analyzer (Applied Biosystems) using POP-7™ Polymer (Applied Biosystems). The KB base caller was applied to analyze sequence trace files. Data (electropherogram) from these ABI sequence trace files were exported using Chromas 2.31 software (Technelysium Pty Ltd.).

### Data Processing

The process of data analysis of the TFF1 gene (forward [G-rich] primer) is exemplarily depicted in FIG. 1 and FIG. 2. Sequence data from the 50% methylated DNA was exported from the “.ABI” trace files and the trace of the C signal (containing the methylation signal and the normalization tail) is depicted (FIG. 1, step 1 and 2). The signals at the CpG sites and the normalization tail were identified and excised (FIG. 1, step 2 and 3).

FIG. 2 (A) shows the C traces of all used standard DNAs (O %, 5%, 10%, 25%, 50%, 75% and 100% methylated). CpG sites and the normalization tail were already excised in FIG. 2. Normalization was performed by dividing the area of all CpG sites by the area of the normalization tail (FIG. 2, B). Quantitative methylation information is reflected by the normalized area (methylation index) of the CpG sites (FIG. 2, C).

TABLE 1
Sequences of oligonucleotides used as primers.

| | | |
|---|---|---|
| Gene-Amplicon | Primer SEQ ID NO | Sequence 5′->3′ |

| | | |
|---|---|---|
| LHX3 | forward primer | actccAGAAGGGTAGGGTTAGTGTTTTT |
| | SEQ ID NO: 1 | |
| | reverse primer | aggtgACCCCTCTAAAACCCAAAATAACC |
| | SEQ ID NO: 2 | |
| PITX3 | forward primer | actccTTTTAGTAGGGTAGTTGGAAAGGG |
| | SEQ ID NO: 3 | |
| | reverse primer | aggtgTACTACCACCCCCAACC |
| | SEQ ID NO: 4 | |
| HS3ST2 | forward primer | actccGGGATTTTTGGAGAAGTTTTTTGGT |
| | SEQ ID NO: 5 | |
| | reverse primer | aggtgCTCCTACACTTACCTATATTACAACT |
| | SEQ ID NO: 6 | |
| SLIT2 | forward primer | actccGTGAGTGAGTAGAGTTTAGAGT |
| | SEQ ID NO: 7 | |
| | reverse primer | aggtgAAAAATCTCAATAAATATTATAACCCC |
| | SEQ ID NO: 8 | |
| SLITRK1 | forward primer | actccGAGGTGATAAATATTAGTAGTAGTTT |
| | SEQ ID NO: 9 | |
| | reverse primer | aggtgAAATTTCATACTCCTCTCCAATAAC |
| | SEQ ID NO: 10 | |
| LIMK1 | forward primer | actccAAGGGAGGTTTGGTGTATTTTTT |
| | SEQ ID NO: 11 | |
| | reverse primer | aggtgAATACCCTATAAACCACCCCCC |
| | SEQ ID NO: 12 | |
| PITX2-4 | forward primer | actccAGAGGGATAAAGAGTAAAGATTTAG |
| | SEQ ID NO: 13 | |
| | reverse primer | aggtgCCAATAACCTCTCCCTATAAC |
| | SEQ ID NO: 14 | |
| PITX2-2 | forward primer | actccTTTTGGAAAGTGGTTTTTAGTTTTTG |
| | SEQ ID NO: 15 | |
| | reverse primer | aggtgCCAAACAACCCAACTCTTCCAC |
| | SEQ ID NO: 16 | |
| TFF1 | forward primer | actccAGTTGGTGATGTTGATTAGAGTTTT |
| | SEQ ID NO: 17 | |
| | reverse primer | aggtgCCCTCCCAATATACAAATAAAAACTACT |
| | SEQ ID NO: 18 | |
| PLAU | forward primer | actccGTTAGGTGTATGGGAGGAAGTA |
| | SEQ ID NO: 19 | |
| | reverse primer | aggtgACTCCCTCCCCTATCTTACAAC |
| | SEQ ID NO: 20 | |
| PITX2-1 | forward primer | actccGTAGGGGAGGGAAGTAGATGTTTAG |
| | SEQ ID NO: 21 | |
| | reverse primer | aggtgTTCTAATCCTCCTTTCCACAATAAAA |
| | SEQ ID NO: 22 | |
| PITX2-3 | forward primer | actccGATAGGTAGGTGATATTAGATTTTTT |
| | SEQ ID NO: 23 | |
| | reverse primer | aggtgCCTAAATACCTAAAACTAAACTAC |
| | SEQ ID NO: 24 | |
| RASSF1A | forward primer | actccAAGGAGGGAAGGAAGGGTAA |
| | SEQ ID NO: 25 | |
| | reverse primer | aggtgTCCCCCAAAATCCAAACTAAAC |
| | SEQ ID NO: 26 | |

All primers contain a gene specific sequence (capital letters) and a normalization tag (lower case letters).

### Results

FIGS. 3 to 15 show the results of the sequencing of the 0%, 5%, 10%, 25%, 50%, 75% and 100% methylated DNA mixtures using 13 PCR amplicons (10 different genes). All amplicons were sequenced using the forward and the reverse primer. The methylation index is displayed by the normalized areas of the peaks of the respective methylation sites, which were normalized against the normalization tail. The methylation indices of all 13 amplicons and both sequencing directions for each amplicon show a strong correlation to the level of methylation of the input DNA. In this case, the methylation index is the sum of all methylation signals within one analyzed nucleic acid, and therefore reflects the average methylation per analyzed sequence. Alternatively, this analysis can also be performed separately for each CpG site.

### Example 2

The new method of quantification of bisulfite sequencing enables quantification of methylation with high resolution. The principle of the new method is depicted in FIG. 18 and is based on the introduction of a normalization signal in the PCR product. The addition of bisulfite drives the conversion of the DNA in that all cytosines are being converted to uracil. Solely methylated cytosine remains unaffected from this reaction. Accordingly, the DNA contains cytosines only where methylated cytosine was previously found (FIG. 18, I). Now this DNA is PCR-amplified whereby one uses a backward-primer, which contains a gene-unspecific domain with guanosines at its 5′ end (FIG. 18, II). Thereby, one generates a strand that contains cytosines at its 3′ end, which originate from the domain of the backward-primer, in addition to the cytosines originating from the methylated cytosines (FIG. 18, III). Thus, only the sequence signal from the cytosine is examined and the other three bases can be ignored. The additional cytosines, at the end of the PCR product, are independent of the original relative methylation and are present in each and every DNA molecule of the PCR product. They behave, therefore, like cytosines, which in the original DNA were methylated up to 100%, and can be used for the normalization of the actual methylation signal.

In FIG. 18, the principle of this method for sequencing with a forward-primer is shown. Since the bisulfite-converted template DNA contains no cytosines besides those in the previously methylated CpG positions, the forward-primer also contains no cytosines but instead is relatively rich in guanosines. Therefore, the direction of the sequencing is referred to in the following as G-rich sequencing. Similarly, the method allows sequencing to be carried out using the backward-primer. This sequencing with the C-rich primer is accordingly referred to in the following as C-rich sequencing. In the case of C-rich sequencing, the normalization signal is accordingly generated in the PCR product in that the G-rich forward-primer contains a domain with some cytosines at its 5′ end.

The signal normalization is shown in FIG. 19. In this figure, the cytosine signals are produced from five different methylated DNA standards (0, 5, 15, 50, and 100% methylation). One can see that without the preceding normalization (FIG. 19, top) no correlation would exist of the peak-height and peak-area of the methylation signals with the methylation of the starting DNA. If, however, one normalizes the signals in the form that the cytosines, artificially inserted into the PCR product, have a uniform value, then the correlation of the peak-height and peak-area of the methylation of the starting DNA can be perceived well (FIG. 19, bottom).

The principle approach to analyze the sequence data using the normalization signals is schematically depicted in FIG. 19 and described in detail in the following. First, the data, which was generated from the sequencing device, are transferred into an appropriate format. The fluorescence intensities of the four dyes of all four bases are contained in this file for each measuring point. Since each measuring point represents a time point, this file can be depicted as fluorescence intensity as a function of (running-) time in the capillary gel electrophoresis. For the analysis, the only information of the fluorescent dyes necessary is that which carries the methylation information. In the case of the G-rich sequencing, it is the C-signal, and in the case of the C-rich sequencing, the G-signal (FIG. 18). The signals from the remaining dyes are not examined further. Now, the methylation-carrying signals from all the samples are depicted in a diagram as shown in FIG. 19. Since there are slight differences from sample to sample in the running patterns during the capillary electrophoresis, these signals along the x-axis (running time axis) are thus shifted such that the normalization signals lie over one another—as in FIG. 19. Now, the normalization is performed. For that, an area of the x-axis is identified that comprises the entire normalization signal. In FIG. 19, this region is from 1900-2050 (running time), for example. The peak-area of the signal in this region is determined in that the fluorescence intensities are summed up (integrated) in this area. The actual normalization is performed in that the fluorescence intensities are divided into each measuring point along the peak-area of the entire normalization signal. As a result, the peak-area of the normalization signal averages to exactly one for each sample after the normalization, and all samples can be directly compared to each other. After this normalization, the actual quantitative methylation information is contained in the height of the peak, corresponding to the methylation positions, as well as in the area below the peak. In FIG. 19, one sees this correlation clearly: After normalization is performed (FIG. 19, bottom), the peak-height as well as the peak-area at the CpG positions correlate very well with the methylation of the introduced DNA standards. In the following, only the peak-areas are used for a further analysis. The reason for this was the consideration that the peak-area is represented by the sum of several measuring points and does not constitute only one measuring point as the peak-height. Thus, the peak-area should, therefore, give a more robust signal than the peak-height. In FIG. 19, it can be seen that each unique CpG position can be separately analyzed. If not otherwise described, then in this example, the sum of the peak-areas is used within each PCR product in order to further increase the robustness of the signal. The definition of the peak-area of a methylation position (CpG position) is defined as follows: Fluorescence of the local maximum plus the fluorescence intensities of the 15 preceding and the 15 subsequent measuring points. A peak is thus the sum of 31 measuring points.

In this example, the methylation of a total of 13 loci is examined from the micro-dissection samples. Since there is obviously not enough DNA present in the micro-dissection material, the DNA is first pre-amplified in a PCR in order to analyze the 13 loci separately from one another. For this purpose, a PCR is performed where the primers for all 13 loci are contained (multiplex-PCR, mPCR). Subsequently, each of the 13 loci is re-amplified in a separate PCR (singleplex-PCR, sPCR) whereby the product of the multiplex-PCR pre-amplification served as template. Primers with the 5′ domains for the generation of the normalization signals are first used in the sPCR re-amplification. The pre-amplification with the multiplex-PCR is performed with conventional primers without domains. In the multiplex pre-amplification, the emphasis was placed on specificity. As a result of relatively low concentrations of MgCl₂ and primers for mPCRs and a high primer-annealing temperature during the PCR, the formation of side products and primer-dimers are hindered despite high numbers of cycles and a low concentration of template. The product of this mPCR pre-amplification is, thus, suited to be re-amplified subsequently in an sPCR without the potential of side products being better amplified than the desired loci. In order to minimize further the formation of side products during re-amplification, the primers being used were those, which through additional bases near the 5′ domain and in the 3′ end differ from pre-amplification primers. As far as it was possible and the primers of pre-amplification did not directly border a CpG, the primers of re-amplification were lengthened up to 3 bases in the amplificate. Thus, it was ensured that only the correct amplificate was reproduced.

The efficiency of the new method was tested for the 13 loci to be investigated. For that purpose, the DNA standards (0, 5, 10, 25, 50, 75, and 100% methylated) are processed according to the described procedure. The DNA standards are composed of a mixture of genome-wide amplified DNA (MDA DNA) and synthetically methylated MDADNA in the corresponding ratio. 20 ng of each bisulfite-converted DNA standards are first pre-amplified in the mPCR and subsequently re-amplified in the 13 separate sPCR's. The PCR product of the re-amplification was sequenced with both G-rich forward-primer as well as the C-rich backward-primer. FIG. 20 shows the sequencing result of the DNA standards. As can be seen, the G-rich and C-rich sequencing leads to the same results for all 13 sequencing assays. In each of these 13 assays, it was possible to distinguish between all investigated methylation standards.

To compare with conventional methods, the methylation for three of the 13 loci (PITX2 promoter AB, uPA and TFF1) was additionally determined using the corresponding QM assays (compare to this WO 2005/098035 as well as the following paragraph). Here, 20 ng of bisulfite-converted DNA from each of the DNA standards was used in each of the three QM assays. Moreover, the measurement with the QM assays were performed as a threefold determination so that altogether 240 ng of each of the DNA standards for the three QM assays were used (3×20 ng for each of the three QM assays) as opposed to only 20 ng for each of the standards for all 13 sequencing assays. The result of the comparison of the techniques is depicted in FIG. 21. In the case of the sequencing method, only the sequencing with the G-rich primer is shown in this diagram.

Each of the QM assays was specifically for the analysis of the gene PITX2 (promoter AB and C), TFF 1, ABHD9 and uPA. The primers and the probes for detection used for these assays are portrayed in Table 1. Here, the following regions of the genome were analyzed (after the ensemble v41): chromosome 4, Region 111777835-111777978 (PITX2 AB), chr. 4, 111763501-111763655 (PITX2 C), chr. 10, 75340750-75340828 (uPA), chr. 21, 42656449-42656529 (TFF1), chr. 19, 15204086-15204233 (ABHD9). All PCRs were performed in 20 ul units and had the following composition: 1×PCR Puffer with passive ROX reference (Eurogentec, B), 1 U HotGoldStar Taq polymerase (Eurogentec, B), 0.2 mmol/l each dNTP (dTTP, dATP, dGTP and dCTP, Fermentas, CDN), 0.625 μmol/l each primer and 0.2 μmol/l each probe (Biomers, D). The magnesium concentration was different for the various assays: 3.5 mmol/l in the TFF1 assay, 3 mmol/l each for both the PITX2 assays and in the uPA assay, as well as 2.5 mmol/l for the ABHD9 assay. The QM assays were performed in the 96-well as also in the 384-well plates. Here, optical PCR plates and optical foils (Applied Biosystems, USA) were used. The QM assays were incubated using the following temperature-time profile: initial activation of the polymerase for 10 min at 95° and 45 cycles with 15 s denaturation (95°) and 60 s annealing and extension, each. The annealing and the extension temperature is for the assays PITX2 (promoter AB) 62° C., PITX2 (promoter C) 62° C., TFF1 58° C., uPA 60° C. and ABHD9 60° C. All the QM assays were performed with a 7900HT Fast Real-Time PCR System (Applied Biosystems, USA) with the Emulation switched “off” and the Ramping Rate at maximum. The fluorescence intensities were recorded during each PCR cycle during the extension and annealing steps. The analysis of the Real-Time PCR was performed with the ABI SDS 2.2 Software (Applied Biosystems, USA). The calculation of the methylation was performed using the following formula:

$$Methylation = \frac{100}{1 + {2}^{( CT ( CG ) - CT ( TG ) )}}$$

with CT(CG) and CT(TG): CT the CG i.e. TG detection probes.

TABLE 2

Primer and probes for detection for the QM assays. All the probes carry
at the 3′ end a quencher-(BHQ-1) and at the 5′ end a reporter-dye (6-
FAM for CG-probes and HEX for TG-probes). The primers and the probes
for detection were purchased from Biomers (D).

| | | | | |
|---|---|---|---|---|
| gene | forward primer | reverse primer | CG-probe | TG-probe |

| | | | | |
|---|---|---|---|---|
| PITX2^{a} | GTAGGGGAGGGAA | TTCTAATCCTCCTT | AGTCGGAGTCGGG | AGTTGGAGTTGGG |
| | GTAGATGTT (SEQ | TCCACAATAA (SEQ | AGAGCGA (SEQ ID | AGAGTGAAAGGAG |
| | ID NO: 27) | ID NO: 28) | NO: 29) | A (SEQ ID NO: 30) |
| PITX2^{c} | GATAGGTAGGTGA | CCTAAATACCTAA | CGACTCCTATTCGA | CCCAACTCCTATTC |
| | TATTAGATTTT | AACTAAACTAC | CCGCCCG (SEQ ID | AACCACCCAAAAA |
| | (SEQ ID NO: 31) | (SEQ ID NO: 32) | NO: 33) | (SEQ ID NO: 34) |
| TFF1 | GATGGTATTAGGA | CCCTCCCAATATAC | CACCGTTCGTAAA | ACACCATTCATAA |
| | TAGAAGTATTA | AAATAAAAACTA | ATCC (SEQ ID | AATCCCCTAAT |
| | (SEQ ID NO: 35) | (SEQ ID NO: 36) | NO:37) | (SEQ ID NO: 38) |
| uPA | GTTTTTTTTAAATT | CCTCCCCTATCTTA | ACCCGAACCCCGC | ACCCAAACCCCAC |
| | TTTGTGAG (SEQ ID | CAA (SEQ ID NO: | GTACTTC (SEQ ID | ATACTTCCACA |
| | NO: 39) | 40) | NO: 41) | (SEQ ID NO: 42) |
| ABHD9 | GGTGTTAGGGTTTA | CCAAATATTTACCT | AACTATTTTCTATC | AACTATTTTCTATC |
| | GGGGTT (SEQ ID | AACACTCAAATA | GAAACCGCCCG | AAAACCACCCACC |
| | NO: 43) | (SEQ ID NO: 44) | (SEQ ID NO: 45) | TCT (SEQ ID NO: 46) |

^{a}promotor of transcript A and B, ^{c}promotor of transcript C

FIG. 21 clearly shows that for both genes PITX2 and TFF1 the corresponding sequence assays and the QM assays deliver comparable results. For the assays for the gene uPA, one sees in FIG. 21 that with the QM assay in the lower methylation region, a resolution is hardly possible. The sequencing assay, in contrast, shows the equivalent good resolution in this region as for the other genes PITX2 and TFF1.

A QM assay yields a value between 0 and 100% due to the method of analysis by which the signals of the two probes are compared to each other. This value is equated, as a simplification, with the percentage of methylation. In contrast to that, the sequencing assays yield values that can be very different from assay to assay. Since this value reflects the sum of the normalized peak-areas at the CpG positions, such a value is dependent, for example, on the number of CpG positions being examined. Furthermore, the fluorescence intensities that a base generates during sequencing are also dependent on the position in the DNA fragment and the surroundings. A methylated cytosine can yield, thus, at one position another signal intensity than a methylated cytosine in another position. In order to conclude on the percentage values of methylation, based on the normalized peak-areas, the analyzed DNA standards were used for the calibration of the 13 assays. Simplified, a linear regression of the data shown in FIG. 20 is performed and on the basis of this regression, the normalized methylation signals are converted to a percentage value of methylation.

For the identification of a biomarker for a certain question, it is sufficient in a majority of the cases to use the normalized intensities (peak-height or peak-areas), since these as criteria can be used readily in order to technically ascertain whether a potential biomarker is able to differentiate between two groups (e.g. patients with either a good or bad prognosis). In order, however, to be able to compare two biomarkers with each other, knowledge of the differences in methylation of the two groups is also necessary (magnitude of effect). A biomarker, for which the difference in methylation of the respective patient groups tends to be larger, can be more suitable than a biomarker with a smaller magnitude of effect. This is particularly true when the difference in methylation is located in a region, which is technically difficult to resolve. In order to be able to make a biological statement about the properties of a biomarker, as for example, in micro-dissected regions of a tumor, the determination of a percentage value of methylation is meaningful. A first approximation of the percentage of methylation can also be decided upon in that the intensities at the corresponding CpG position can be compared with the intensity of a normalization signal, which corresponds to 100% methylation. However, this is less accurate than a calibration, since the signal intensities decrease toward the end as a rule within the sequence histogram and are also dependent on the individual position and the environment of the CpG position. For an exact calculation of the percentage value of methylation, a calibration standard is, therefore, necessary based on which the normalized fluorescence intensities can be calibrated.

When comparing the QM method with the quantitative sequencing, one should consider that each of these yield somewhat different information. Since the QM method is based on detection probes, which cover several CpGs, only such DNA molecules should be detected where the CpGs are either consistently methylated or consistently unmethylated (co-methylation). By the above-analyzed DNA standards, this is irrelevant because these DNA standards are made out of completely methylated and completely unmethylated DNA, and so, co-methylation is present. Therefore, the QM method and sequencing assays on DNA standards are directly comparable. In addition, an average value was formed over the entire amplificate when analyzing the sequence histograms so that this value also contains information that is not covered by the QM assay.

In the following, the convertibility and usability of the sequencing methods that were developed and established with artificially methylated DNA standards were analyzed on clinical samples. For each of 13 different patients, ten sections (10 um) of formalin-fixed paraffin-embedded breast cancer tumors were lysed and the DNA extracted. After bisulfite treatment of these DNAs, they were treated as described above, i.e., 20 ng of each patient DNA was pre-amplified in a mPCR. Subsequently, each of the 13 amplificates was re-amplified in a sPCR and then sequenced. Sequencing was performed both with the G-rich forward as well as with C-rich backward-primer. Sequencing data were analyzed as described above and the resulting normalized methylation signals were calibrated based on the standards described above (FIG. 20).

First, the results of the C-rich and G-rich sequencing were compared. As already shown for the DNA standards analyzed above, the results of these two sequencing analysis correlate very well with each other (regression co-efficient R²=0.96).

A quantitative methylation analysis of many biomarkers in small amounts of tissue has thus far hardly been possible. This is mainly due to the fact that quantitative methods as e.g. Real-Time PCR, need a relatively large amount of template DNA for a robust result. Therefore, the template DNA was first pre-amplified in a mPCR. Then the product of this pre-amplification was re-amplified in individual sPCRs and analyzed. For this analysis, the method according to the invention of quantitative bisulfite sequencing was used, which allows for the determination of the methylation of individual CpG positions with high resolution.

This new sequencing method is based on the incorporation of an artificial methylation signal in the PCR product through modified primers. Based on these signals, an internal normalization is performed. The advantages of the method according to the invention compared to methods known from the state of the art are discussed in the following.

This sequencing of bisulfite-treated DNA is a powerful method for the analysis of DNA methylation. Until now, three different methods were used for this: direct sequencing according to Sanger, sequencing of clones, and pyro-sequencing.

When sequencing directly according to Sanger, the PCR product from a sample is directly sequenced (Grunau C, Clark S J, Rosenthal A (2001). Bisulfite genomic sequencing: systematic investigation of critical experimental parameters. Nucleic Acids Res 29(13):E65-5). In order to extract quantitative methylation data from the sequence histogram, a comparison is made of a CpG position measured C-signal (originally a methylated cytosine) with the corresponding T-signal (originally unmethylated cytosine) (Lewin J, Schmitt A O, Adorjan P, Hildmann T, Piepenbrock C (2004). Quantitative DNA methylation analysis based on four-dye trace data from direct sequencing of PCR amplificates. Bioinformatics 20(17):3005-12). Thereby, this comparison is formed such that the C-signal is divided by the sum of the T-signal and C-signal and the outcome is a percentage value of methylation. A basic problem results from this approach, and that is, that the resolution capacity of this method is considerably impaired. During the sequencing or during the recording of the sequence histogram, the four dyes, which are specific for the four bases, are measured independently of each other. Subsequent to that, the data is further processed. For that, an algorithm is used (the so-called “Basecaller”), which scales these four independent signals against each other. For one, the running differences of the four different bases are equalized in that newly scaling is performed along the x-axis; for another, the intensity differences are also compensated for in that the four signals are also scaled along the y-axis. The latter causes the low theoretically possible resolution capacity, since for an accurate quantification an exact scaling of the T-signal to the C-signal is necessary. Since only the signals from the originally methylated cytosine still exist in the C-signal, the exact scaling is difficult. The sequence histogram of unmethylated DNA, for example, contains absolutely no cytosine, which could be used for scaling. Strictly speaking, the scaling can then only precisely take place when the methylation of a sample was known. For an accurate determination of the methylation, the exact value is thus necessary, which is to be determined. Han et al. 2006 (Han W, Cauchi S, Herman J G, Spivack S D (2006). DNA methylation mapping by tag-modified bisulfite genomic sequencing. Anal Biochem 355(1):50-61) recommend to introduce artificially a C-signal using modified primers into the PCR product in order to facilitate the scaling of the two signals (C to T). Nevertheless, according to Han et al. 2006, the problem of accurate scaling, however, still exists.

In contrast to that, in the method developed here, the artificially introduced methylation signal, which is introduced through the primer, is directly used for normalization. For this reason, an accurate scaling of the C to T signal is not necessary because the T-signal for a determination of methylation is not considered.

The problem described of the correct scaling of both the interesting signals of the sequence histogram can also be bypassed, if, instead of the direct sequencing, a clone is sequenced. Here, a PCR product is generated starting from a bisulfite-treated DNA sample that is cloned, and single clones are sequenced. According to this sequencing method, either only a C-signal or a T-signal can occur at the CpG positions, but no mixtures of both signals and therefore, the analysis is unequivocal. However, in order to be able to make conclusions about the methylation status of the complex sample, it is necessary to sequence a large number of clones stemming from this sample. Thereby, this method is work-intensive, expensive, and time consuming.

The third sequencing method is the pyro-sequencing. In this method, sequencing is done according to the principle “sequencing through synthesis.” The four different dNTP's are alternately added into the mixture and the introduction of each of the corresponding nucleotide is quantified through a biochemical measurement of arising pyrophosphates. Via the number of introduced cytosines and introduced tyrosines in a CpG position, the methylation can be ascertained. One disadvantage of this method is that only amplificates 100-200 bp long can be sequenced (Ronaghi M (2001). Pyrosequencing sheds light on DNA sequencing. Genome Res 11(1):3-11) and in the case of bisulfite-treated DNA, actually only about 80-100 bp with a maximum of 10-15 CpG positions (Tost J, Gut I G (2006). Analysis of gene-specific DNA methylation patterns by Pyrosequencing® technology. Methods Mol Biol 373:89-102, Brakensiek K, Wingen L U, Langer F, Kreipe H, Lehmann U (2007). Quantitative high-resolution CpG island mapping with Pyrosequencing reveals disease-specific methylation patterns of the CDKN2B gene in myelodysplastic syndrome and myeloid leukemia. Clin Chem 53(1):17-23). The amplificates analyzed in this example, with the quantitative bisulfite sequencing method according to the invention, had an average length of 169 bp and contained an average of 12 CpGs. Thereby, the length and the CpG frequency of these amplificates were not optimized for this method so that presumably there remains a potential for sequencing longer amplificates with a higher number of CpGs. For this reason, this method according to the invention for quantitative bisulfite sequencing is more powerful than the pryro-sequencing. Moreover, it posses a higher resolution capacity in particular at low methylation level. According to the state of the art of pyro-sequencing, the measured methylation values between 0% and 5% are defined as background signal (Shaw R J, Liloglou T, Rogers S N, Brown J S, Vaughan E D, Lowe D, Field J K, Risk J M (2006). Promoter methylation of P16, RARbeta, E-cadherin, cyclin A1 and cytoglobin in oral cancer: quantitative evaluation using pyrosequencing. Br J Cancer 94(4):561-8, Jones A V, Kreil S, Zoi K, Waghorn K, Curtis C, Zhang L, Score J, Seear R, Chase A J, Grand F H, White H, Zoi C, Loukopoulos D, Terpos E, Vervessou E C, Schultheis B, Emig M, Ernst T, Lengfelder E, Hehlmann R, Hochhaus A, Oscier D, Silver R T, Reiter A, Cross N C (2005). Widespread occurrence of the JAK2 V617F mutation in chronic myeloproliferative disorders. Blood 106(6):2162-8, Brakensiek et al. 2007 see above). In contrast with the new method according to the invention for quantitative bisulfite sequencing, a distinction between 0% and 5% is highly possible. Thus, a quantification is possible in this methylation region. This demonstrates precisely for the analysis that a lowly methylated biomarker has a decided advantage in comparison to pyro-sequencing.

### Example 3

### Detection of the Clonality of a Sample

Tail tagged primers can also be used to analyse the state of clonality of a sample. Therefore, a loci is chosen which comprises at least one methylation site and a length polymorphism (i.e. short tandem repeat, STR). In addition, one allele of the loci is silenced by DNA methylation, whereby this silencing occurs randomly. Genes, which fulfill these criteria can be found i.e. on the X-chromosome which is randomly inactivated. As depicted in FIG. 22, the sequencing of such a loci using tag tailed primers can be used to determine, whether a sample consists of cells from the same or from different progenitor cells. A gene which is located on the X-chromosome and which comprises a STR and methylation sites is the human androgen receptor (SEQ ID NO: 47 5′ggccccaggacccagaggccgcgagcgcagcacctcccggcgccagtgctgctgctgcagcagcagcagcagcagc agcagcagcagcagcagcagcagcagcagcagcagcagcagcagcagcaagagactagccccaggcagcagcagcagca gcagggtgaggatggttctccccaagcccatcgtagaggccccacaggctacctggtcctggatgaggaac3′). The following primers can be used to amplify this region on bisulfite converted DNA and to incorporate the tail: SEQ ID NO: 48 5′cgtcgtcgaaccccaaacacccaaa3′ and SEQ ID NO: 49 5′gttttttaggattaggtagtttgt3′, where cgtcgtcg reflects the tail.

Accordingly, the clonality of tumor can be determined. Therefore, genomic DNA is isolated from cancerous cells and converted by means of bisulfite. Subsequently the bisulfite converted DNA is sequenced according to the method of the invention. Wherein only either the maternal or the paternal allele is detected, the tumor is of monoclonal origin. Wherein both the maternal and paternal allele are detected, the tumor is of polyclonal origin.

### DEFINITIONS

The term “base caller” as used herein refers to, but is not limited to, an algorithm used to process the sequencing raw data obtained directly from the detection of the fluorescence signal(s) over time. During a sequencing run, the signals of all four bases are detected independently from another. Subsequently, the said algorithm scales all signals along the x-axis, whereby the differences in the running behaviour of the different bases are compensated. In addition, also the signals are scaled along the y-axis. Thereby differences in the signal intensities are compensated.

The term “methylation index” as used herein refers to, but is not limited to, the absolute amount of methylation at an analyzed CpG dinucleotide after normalization. Therefore the raw sequencing data is normalized to the signal(s) of the introduced base resulting in normalized signal intensity. The term “methylation index” may refer, but is not limited to, the peak area or peak height or the sum of a multiply peak areas or heights of several CpG dinucleotides.

The term “methylation rate” as used herein refers to, but is not limited to, the percentage of methylation at an analyzed CpG dinucleotide. Therefore the methylation index is trans-formed into a percentage methylation based on a calibration curve.

1. A method for determining the methylation rate of a nucleic acid through sequencing, comprising the steps of:
treating the nucleic acid with a chemical reagent or an enzyme containing solution, whereby the base pairing behavior of methylated cytosine bases and/or unmethylated cytosine bases of the nucleic acid are altered such that methylated cytosine bases become distinguishable from unmethylated cytosine bases, and
introducing into the nucleic acid at least one base for generating a sequencing signal to be used as a reference signal for normalization, and
sequencing the nucleic acid, whereby a signal from each
cytosine base of the nucleic acid, or
guanine base of the nucleic acid
and a reference signal from the at least on introduced base is obtained, and
normalizing the signal obtained from each
cytosine base of the nucleic acid, or
guanine base of the nucleic acid
to the reference signal from the at least one introduced base.

2. The method according to claim 1, wherein the at least one introduced base is:
at least one base analog, and/or
at least one cytosine base, and/or
at least one guanine base.

3. The method according to claim 1, wherein the nucleic acid is treated with a bisulfite containing solution, whereby unmethylated cytosine bases of the nucleic acid are converted into sulfon-uracil bases or uracil bases whereas methylated cytosine bases remain unchanged.

4. The method according claim 1, wherein the at least one base is introduced into the nucleic acid through an amplification reaction and/or a ligation reaction.

5. The method according to claims 1, wherein the at least one signal stemming from the at least one introduced base is identified.

6. The method according to claim 1, wherein normalization occurs either by dividing the area value of each signal stemming from a cytosine base or a guanine base of the nucleic acid by the area value of the signal from the at least one introduced base, or by dividing the height value of each signal stemming from a cytosine base or a guanine base of the nucleic acid by the height value of the signal from the at least one introduced base.

7. The method according to claim 6, wherein percentage of methylation of a specific position is obtained by calibrating the normalized signal of the nucleic acid to be analyzed against the normalized signal of a reference nucleic acid.

8. A method for determining the clonality of a sample, comprising
a) isolating genomic DNA from a sample;
b) submitting the isolate genomic DNA to the method of claim 1, wherein normalized signals for cytosine bases or guanin bases are obtained,
c) deducing that a sample is of monoclonal origin wherein only cytosine bases or guanine bases are detected that are specific for either the maternal chromosome or the paternal chromosome; or deducing a sample is of polyclonal origin, wherein cytosine bases or guanine bases are detected that are specific for the maternal as well the paternal chromosome.

9. An oligonucleotide for amplifying a nucleic acid, comprising
a first sequence part that is reverse complementary to the nucleic acid to be amplified for initiating an amplification reaction,
a second sequence part that contains at least one base for generating a sequencing signal to be used for the normalization of sequencing signals stemming from the nucleic acid, and
a third sequence part for the hybridization of a sequencing primer.

10. A kit for the realization of the method according to one of claims 1 to 9, with the following components:
a) a chemical reagent or an enzyme which alters the base pairing behavior of methylated cytosine bases and/or unmethylated cytosine bases of the nucleic acid such that methylated cytosine bases become distinguishable from unmethylated cytosine bases,
b) at least one oligonucleotide that comprises at least one cytosine base and/or at least one guanine base and/or at least one base analog, and
c) an enzymatic activity for amplifying a nucleic acid using the at least one oligonucleotide as a primer and/or an enzymatic activity for ligating the at least one oligonucleotide to a nucleic acid.
